SEQUENCE LISTING (21 sequences)

SEQ ID NO: 1 - DNA, 805 bp, Homo sapiens
gccaggaggcggagtggaagtggccgtggggcgggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggtgggagatgccatcccagcagtggaggtgtttgaaggggagccagggaacaaggtgaacctggcagagctgttcaagggcaagaagggtgtgctgtttggagttcctggggccttcacccctggatgttccaagacacacctgccagggtttgtggagcaggctgaggctctgaaggccaagggagtccaggtggtggcctgtctgagtgttaatgatgcctttgtgactggcgagtggggccgagcccacaaggcggaaggcaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccagatttctgcaataaacacttgtggtttgcggaaaaaaa

SEQ ID NO: 2 - protein, 162 aa, Homo sapiens
MAPIKVGDAIPAVEVFEGEPGNKVNLAELFKGKKGVLFGVPGAFTPGCSKTHLPGFVEQAEALKAKGVQVVACLSVNDAFVTGEWGRAHKAEGKVRLLADPTGAFGKETDLLLDDSLVSIFGNRRLKRFSMVVQDGIVKALNVEPDGTGLTCSLAPNIISQL

SEQ ID NO: 3 - DNA, 780 bp, Rattus rattus
tgcgtcctaggcagcatagccggatcggtgctccgtgcatcggctacttggacgtgcgtggcaggcagagcaggccggaaaggagcaggttgggagtgtggtggggcccgcagcttcagcagtgccgcggtgactatggccccgatcaaggtgggagacaccattccctcagtggaggtatttgraggggaacctggaaagaaggtgaacttggcagagctgttcaaggacaagaaaggtgttttgtttggagtccctggggcatttacacctggctgttccaagacccatctgcctgggtttgtggagcaagccggagctcygaaggccaagggagcacaagtggtggcctgtctgagtgttaatgatgycttcgtgactgcagagtggggtcgagcccaccaggcagaaggcaaggttcagctcctggctgaccccactggagcttttggaaaggagacagatttactactagatgattctttggtgtctctctttgggaatcgtcggctaaaaaggttctccatggtgatagacaagggcgtagtaaaggcactgaacgtggagccggatggcacaggcctcacctgcagcctggcccccaacatcctctcacaactctgaggccctgaccagaatgtcctctgactctcccatctcctccacccagctctgggccaaaggcccagtacctccttacctgagggccactggaatggaaccttgacaatatttctgcaataaacagtttaatttgtgaaaaaaaaaaaaaaaaaaaaa

SEQ ID NO: 4 - protein, 162 aa, Rattus rattus
  MISC_FEATURE (17)..(17): X = E or G
MAPIKVGDTIPSVEVFXGEPGKKVNLAELFKDKKGVLFGVPGAFTPGCSKTHLPGFVEQAGAXKAKGAQVVACLSVNDXFVTAEWGRAHQAEGKVQLLADPTGAFGKETDLLLDDSLVSLFGNRRLKRFSMVIDKGVVKALNVEPDGTGLTCSLAPNILSQL

SEQ ID NO: 5 - DNA, 675 bp, Mus musculus
tgctccgtgcatcgacgtgcttggcaggcagagcaggccggaaagaagcaggttgggagtgtggcggagcccgcagcttcagcagctccgcggtgaccatggccccgatcaaggtgggagatgccattccctcagtggaggtatttgaaggggaaccgggaaagaaggtgaacttggcagagctgttcaagggcaagaaaggtgttttgtttggagtccctggggcatttacacctggctgttctaagacccacctgcctgggtttgtggagcaagctggagctctgaaggctaagggagcgcaggtggtggcctgtctgagcgttaatgacgtctttgtgattgaagagtggggtcgagcccaccaggcagaaggcaaggttcggctcctggctgaccccactggagcctttgggaaggcgacagacttattattggatgattctttggtgtctctctttgggaatcgtcggctgaaaaggttctccatggtgatagacaacggcatagtgaaggcactgaacgtggagccagatggcacaggcctcacctgcagcctggcccccaacatcctctcccaactctgaggccctggccagatgtcctctgactctcccatctctcccacccggctctaggccaaaaggctcggtacctccttactgggagccacgt

SEQ ID NO: 6 - protein, 162 aa, Mus musculus
MAPIKVGDAIPSVEVFEGEPGKKVNLAELFKGKKGVLFGVPGAFTPGCSKTHLPGFVEQAGALKAKGAQVVACLSVNDVFVIEEWGRAHQAEGKVRLLADPTGAFGKATDLLLDDSLVSLFGNRRLKRFSMVIDNGIVKALNVEPDGTGLTCSLAPNILSQL

SEQ ID NO: 7 - DNA, 469 bp, Homo sapiens
gggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccgtgtgctggggccctgcaattggaatgttggccag

SEQ ID NO: 8 - DNA, 601 bp, Homo sapiens
gggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaagacacacctgccagggtttgtggagcaggctgaggctctgaaggccaagggagtccaggtggtggcctgtctgagtgttaatgatgcctttgtgactggcgagtggggccgagcccacaaggcggaaggcaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccag

SEQ ID NO: 9 - DNA, 604 bp, Homo sapiens
gggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggtgggagatgccatcccagcagtggaggtgtttgaaggggagccagggaacaaggtgaacctggcagagctgttcaagggcaagaagggtgtgctgtttggagttcctggggccttcacccctggatgttccaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccag

SEQ ID NO: 10 - DNA, 2710 bp, Homo sapiens
tctgtcccttagcgcccccgcgggggcttaccccatcccactccatgacctcccctccccccatggcgaattcccacctttctgtctttcactcacttcctggaaccgtccccagggccttggaccttcccccttctcctcccaaaccttgtgagaccccattccctttctacttcatcctgctctcaacttttgggctcctcagaggccctcacccctgactctctctccctacccactctggtcccatgaagccctcaagtactctggggatggatccttcccccttcaaaagattccttcttttgttctacacctcctgggtgtaggggcctggacaccctcccccaacgttccacctgccgctgcccttcctcttcctcctcctgagggtgggaccctcagacctggccaagatcctctccctccatgttgtcagggactcctcctcacccccaaatacagccctctagcccctgtccattttattccactcctttcctgtaacctagacagcatgttatgcaaccctttgcgacacatggggaaaccttccctcccttcctctgttgtcaccaatggccccttaagaggagcagggccaccttgaaacttggaggatatggggtaacccagtgggagcgggcagggagggcccttggaaactgacagggctggagtatcctgctgggtttcagccccggttcctgcaggcacagctgccaggctctctgttcaccttcctgcctctggtttgccccggctccctcaccccccttaccctggagtccttccttctaggtgggagatgccatcccagcagtggaggtgtttgaaggggagccagggaacaaggtgaacctggcagagctgttcaagggcaagaagggtgtgctgtttggagttcctggggccttcacccctggatgttccaaggtgaggcccttccccttctgaagatcaggacctggggatcttttgtgttgctcttaagtcctccacatagtcctgataggactcctaaaaagcatttcagtgccatcacaaaacaagtagagctgggtagagctgggcgcggtggctcacgcctgtaatcccagcactttgggaggccaaggcgggtggatcacgaggtcaggagtccaaaaccagcctggccaagatggtgaaaccctgtctctactaaaaatgcaaaaaaatcagccggatatggtggcgggcgcctgtaatcccaggtattggggaggctgaggcagagaattgcttgaacccaggaggcgtaggttgcagtgagtggagatcgtgcctctgcagtccagcctgggtgaaagagcgagactccgtctcaaaatgaaaaaaaaaaaagaaaacaagtagagactgcaaaaagggaacagtaccgggaatgttggagaaaaacatactacaattaaatccaacacccctgttggtcctgctaaatgacaggcactgtggaaggtgcttgggactcagataaataagacaaagatctgcccatggaaagttcacgtctggaccataaggcattaggtttcattctgagcttcctagtggccaaggcaaaaaggaaatagaatggtttagacagctctcattgtctgatcaaaggtgttgaggcagagcactgaggagggcctggagataaagggtgggctgggggtcagatgcagttatccctttgccgaccctttgttccccttcctcagacacacctgccagggtttgtggagcaggctgaggctctgaaggccaagggagtccaggtggtggcctgtctgagtgttaatgatgcctttgtgactggcgagtggggccgagcccacaaggcggaaggcaaggtgaggtgaggggcctgcagggagtcaggaccaggtaggatattcttcttgtgacctctactttctctgcaggttcggctcctggctgatcccactggggcctttgggaaggtgagtgttcccctgaccgccacagggacatggcggtgcggggagcagtgggggcccttggcctcttcaaggatttctgacacttttctctgtctcttcttaggagacagacttattactagatgattcgctggtgtccatctttgggaatcgacgtctcaagaggtaaaagtggagagtcctctgtggagaaagtcctctgtgggagagagtcctctgtgggagagagtcctctgtggagagggtcctctgtgggaagagtcgtctgtgggggagatgtgtgggagagagtcctgtgtggggagagtcttctgtaggggagagtcctctggggagagagtcctgtgtgggggagagtcctctgtggggagagtcctctgtgtggagagagtcctgtgtggtggtgagtcctctgtgggggagagtcctctgtggggggagtcctctctggagttctcttgggcccctggctgttcactgcctgtctccatgcccagcctccaagcccaggctgatgcagctggctgggcccctctttccggcaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccagat

SEQ ID NO: 11 - DNA, 25 bp, Artificial
  primer
gccatcccagcagtggaggtgtttg

SEQ ID NO: 12 - DNA, 24 bp, primer
ttgaacagctctgccaggttcacc

SEQ ID NO: 13 - DNA, 24 bp, primer
tggaggtgtttgaaggggagccag

SEQ ID NO: 14 - DNA, 24 bp, primer
caggttcaccttgttccctggctc

SEQ ID NO: 15 - DNA, 20 bp, primer
gggtatgggactagctggcg

SEQ ID NO: 16 - DNA, 22 bp, primer
ctggccaacattccaattgcag

SEQ ID NO: 17 - DNA, 24 bp, primer
atgttatgcaaccctttgcgacac

SEQ ID NO: 18 - DNA, 24 bp, primer
gtgtttgaaggggagccagggaac

SEQ ID NO: 19 - DNA, 24 bp, primer
agagacagggtttcaccatcttgg

SEQ ID NO: 20 - DNA, 805 bp, Homo sapiens; identical to SEQ ID NO: 1

SEQ ID NO: 21 - protein, 214 aa, Homo sapiens
MGLAGVCALRRSAGYILVGGAGGQSAAAAARRCSEGEWASGGVRSFSRAAAAMAPIKVGDAIPAVEVFEGEPGNKVNLAELFKGKKGVLFGVPGAFTPGCSKTHLPGFVEQAEALKAKGVQVVACLSVNDAFVTGEWGRAHKAEGKVRLLADPTGAFGKETDLLLDDSLVSIFGNRRLKRFSMVVQDGIVKALNVEPDGTGLTCSLAPNIISQL

(19) United States
(12) Patent Application Publication
(10) Pub. No.: US 2005/0142126 A1
(43) Pub. Date: Jun. 30, 2005

(54) Peroxisome-associated polypeptide, nucleotide sequence encoding said polypeptide and their uses in the diagnosis and/or the treatment of lung injuries and diseases, and of oxidative stress-related disorders

(76) Inventors: Bernard Knoops, Cernoux-Mousty (BE); Cedric Hermans, (US); Alfred Bernard, Brussels (BE); Ruddy Wattiez, Siloupt (BE); Paul Flamagne, Mesvin (BE); Frank Plaisant, Paris (FR); Pierre Gressens, Paris (FR); George A.G. Murrell, Sydney (AU); Min-Xia Wang, Sydney (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/686,157

(22) Filed: Oct. 15, 2003

Related U.S. Application Data
(63) Continuation-in-part of application No. 09/486,167, filed on Aug. 15, 2000, now Pat. No. 6,759,194.

(30) Foreign Application Priority Data
Aug. 20, 1997 (BE) BE 9700692

(51) Int. Cl. C12N 9/02; A61K 38/44

(52) U.S. Cl. 424/94.4; 435/189

(57) ABSTRACT

A peroxisome-associated polypeptide and a nucleotide sequence encoding the peroxisome-associated polypeptide are disclosed. The protein is useful in the diagnosis and/or treatment of several diseases, particularly lung injuries and diseases as well as oxidative stress-related disorders, particularly neurotoxic injury or exitotoxic injury.

### RELATED APPLICATIONS

This application is a continuation-in-part of U.S. application Ser. No. 09/486,167, filed Aug. 15, 2000 which is the U.S. National Phase under 35 U.S.C. § 371 of International Application PCT/BE98/00124, filed Aug. 20, 1998 which claims priority of Belgian application BE 9700692, filed Aug. 20, 1997. Each of the above applications is incorporated herein by reference.

### FIELD OF THE INVENTION

The present invention is related to a new peroxisome-associated polypeptide, the nucleotide sequence encoding said polypeptide and portions thereof as well as their uses in the diagnosis of several diseases, especially the diagnosis and/or the treatment of lung injuries and diseases, and of oxidative stress-related disorders, including neurotoxic injury or excitotoxic injury.

### BACKGROUND OF THE INVENTION

The peroxisomes are organelles nearly ubiquitous in eukaryotic cells. They contain enzymes essential for various catabolic and anabolic pathways. Some of these enzymes are expressed constitutively while others can be induced under appropriate conditions. Peroxisomes carry out a variety of essential reactions such as peroxisomal oxidation and respiration, fatty acid beta-oxidation, cholesterol and dolichol metabolism, ether-phospholipid synthesis, and glyoxylate and pipecolic acid metabolism.

The peroxisomal respiratory pathway is based upon the formation of hydrogen peroxide by a collection of oxidases and the decomposition of the H2O2 by catalase.

These reactions are responsible for 20% of oxygen consumption in liver, and several oxidases have been identified in peroxisomes. Ethanol elimination via catalase in peroxisomes may be significant in addition to the oxidation via cytosolic alcohol dehydrogenase.

The peroxisomal beta-oxidation system catalyses the beta-oxidative chain shortening of a specific set of compounds which can not be handled by mitochondria: very long chain fatty acids, di- and trihydroxycholestanoic acids, pristanic acid, long chain dicarboxylic acids, several prostaglandins, several leukotrienes, 12- and 15-hydroxyeicosatetraeonic acid, and several mono- and polyunsaturated fatty acids, which are of direct diagnostic relevance for some peroxisomal disorders.

Peroxisomes play also a major role in the synthesis of cholesterol and other isoprenoids. Fibroblasts from patients affected by disorders of peroxisome biogenesis show low capacity to synthesize cholesterol.

Two enzyme activities responsible for introduction of the characteristic ether linkage in ether-linked phospholipids (dihydroacetonephosphate acyltransferase (DHAPAT) and alkyldihydroxyacetonephosphate synthase (alkyl-DHAP synthase)) are localized in peroxisomes. These enzymes are not yet cloned. As demonstrated by the identification of patients with deficiency of either DHAPAT or alkyl-DHAP synthase with severe clinical abnormalities, ether-phospholipids are of major importance in humans.

Peroxisomes are able to detoxify glyoxylate via alanine/glyoxylate aminotransferase. The deficiency of this cloned enzyme causes hyperoxaluria type I.

L-pipecolate is a minor metabolite of L-lysine and is catabolized by the L-pipecolate oxidase localised in peroxisomes. The enzyme is deficient in cerebro-hepatorenal (Zellweger) syndrome.

In human, the importance of peroxisomes was emphasised by a number of inherited diseases involving either a defect in the biogenesis of peroxisomes or a deficiency of one (or more) peroxisomal enzymes. So far, 12 different peroxisomal disorders have been described and most of them are lethal.

A wide variety of chemicals have been shown to produce peroxisome proliferation and induction of peroxisomal and microsomal fatty acids-oxidizing enzymes activities in rats and mice. Several peroxisomes proliferators have been shown to increase the incidence of liver tumors in these species. Proposed mechanisms of liver tumor formation by peroxisomes proliferators include induction of sustained oxidative stress.

Therefore, newly identified molecules associated with peroxisomes could be used for the development of diagnostic tools and possibly for the improvement of several therapeutical applications of various diseases associated with peroxisomal disorders. In addition, it is useful to identify the molecules present in specific organs, like the lung, and which may be used as specific markers of inflammatory diseases as well as lung injuries or diseases.

Osteoarthritis (OA) is the most common form of arthritis and is characterized by chronic pain and significant disability. Although the aetiology of OA is multifactorial, the key pathological feature of OA is articular cartilage degradation. The pathological processes of cartilage degradation, including the molecular processes that impair cartilage homeostasis, are still largely unknown. Although there is information regarding the pathological role of reactive oxygen species (ROS) in cartilage degeneration, little is known about the antioxidant defense system and particularly about any ROS scavengers in articular cartilage. The redox status of human chondrocytes in OA is also largely undetermined.

Hence, there is a need in the art to determine the role of the antioxidant defense system, such as the role of peroxidases, in the pathogenesis of OA. Furthermore, there exists a need to treat or prevent OA.

Injury to the perinatal brain is a leading cause of death and disability in children. Partly due to the fact that survival of very pre-term infants has been largely improved in the last decades, neurological handicap of perinatal origin (cerebral palsy) is not decreasing in Western countries and has even been shown to increase in some countries (Hagberg et al. 1996 Acta Paediatr 85:954-60). At the present time there are no therapeutic regimens that are successful for the treatment of injury to the developing brain. Effective research strategies addressing these issues will, therefore, have untold ramifications as affected children have a reduced lifetime potential, families have emotional and financial burdens, and society makes a staggering commitment of resources for their care.

In recent years, the causes of CNS (central nervous system) injury in human neonates have been considered by many to be multifactorial (Nelson et al. (1986 N. Engl. J. Med. 315:81-86; Dammann et al. 2000, Curr. Opin. Pediatr. 12:99-104). Many pre-conceptional, prenatal and perinatal factors (like hypoxic-ischemic insults, endocrine imbalances, genetic factors, growth factor deficiency, abnormal competition for growth factors, maternal infection yielding excess cytokines and other pro-inflammatory agents) are operative in causing injury to the developing brain. For several of these risk factors, excess release of excitatory amino acids and excess free ROS production could represent two major and intricate molecular pathways underlying perinatal brain lesions (Johnston 2001 Ment Retard Dev Disabil Res Rev 7:229-34; Inder et al. 2002 J Pediatr 140:617-21).

Accordingly, there is a need to treat, ameliorate and/or prevent CNS injury caused by oxidative stress.

### SUMMARY OF THE INVENTION

The Inventors have isolated and purified a new sequence of a low molecular weight human polypeptide. Said mammal, preferably human, protein or polypeptide (hereafter identified as peroxiredoxin 5, PRDX5, formerly also known as B18 protein) has been sequenced and its corresponding genomic DNA (SEQ ID NO: 8) and cDNA (SEQ ID NO: 1) have been identified. Similarly, the corresponding nucleotide and amino acid sequence from a rat (SEQ ID NOs: 3 and 4) and from a mouse (SEQ ID NOs: 5 and 6) have been obtained.

Moreover, the nucleotide (SEQ ID NO: 20) and amino acid sequence (SEQ ID NO: 21) of a long form of human PRDX5 have been provided. SEQ ID NO 21 contains a mitochondrial targeting sequence (MTS), which is absent in the short form.

In particular, the long form of human PRDX5 cDNA contains two ATG initiation codons, giving a long or a short PRDX5 form. The short form contains a peroxisomal targeting signal type 1 (PTS 1) and is localized to peroxisomes, the cytosol and the nucleus, while the long form has a mitochondrial localization, due to the presence of a MTS, which is a targeting sequence stronger than the PTS 1. The numbering in the following is based on the amino acid sequence numbering starting at the first Methionine of SEQ ID NO 2 or the first nucleotide, i.e. “G”, of SEQ ID NO 1 (PRDX5), respectively, unless noted differently. The term “PRDX5” may be used to denote an activity similar to the activity by the amino acid sequence of SEQ ID NO: 2. The term does not relate specifically to a length of an amino acid sequence.

Said sequences present several homologies with other peroxisomal proteins of yeast and comprise a carboxy-terminal tripeptide SQL which is necessary for the specific targeting and translocation of several proteins into the peroxisome.

Therefore, the present invention is related to a new isolated and purified polypeptide sequence having a amino acid sequence which presents more than 70% homology, advantageously more than 85% homology, more preferably more than 95% homology, with the amino acid sequence SEQ ID NO: 2. Said amino acid sequence is advantageously obtained from a mammal, preferably from a rat, a mouse or a human.

The present invention is also related to the isolated and purified polypeptide sequence corresponding to the amino acid sequence SEQ ID NO: 2 or a portion thereof, preferably an immunoreactive portion (putative immunogenic domain or T or B cell epitopes).

Said portions are advantageously comprised between:
Glutamic acid position 13—Glutamic acid position 27
Alanine position 26—Leucine position 36
Alanine position 42—Glutamic acid position 57
Glutamic acid position 57—Valine position 69
Valine position 80—Leucine position 97
Arginine position 95—Leucine position 112
Serine position 118—Serine position 129
Valine position 137—Threonine position 150.

Preferably, said portion has more than 10, 20, 30, 50 or 70 amino acids. Specific portions of the amino acid sequence SEQ ID NOs: 2 or 21 are also portions of more than 70 amino acids which present at least 80% of the proteinic activity (see example 5) of the complete SEQ ID NO: 2 sequence. Therefore, the amino acid sequence according to the invention can be partially deleted while maintaining its activity, preferably its anti-oxidative activity, which will be described hereafter.

According to the invention, the amino acid sequence SEQ ID NO: 2 presents a pI of 7.16 and a molecular weight of 17047 Dalton as hereafter defined by bidimensional electrophoresis.

The skilled artisan will appreciate that the amino acids of the present invention may be produced in prokaryotic cells, including bacteria, such as Escherichia coli, or eukaryotic cells, such as yeast, including Saccharomyces cerevisae, or mammalian cells, including CHO-KI cells, or reptiles, birds, amphibians, and fish (see e.g. Sambrook et al. infra).

Accordingly, the present invention relates to the amino acid sequences according to the invention, produced in yeast.

The amino acid sequences according to the invention protect against oxidative stress. Oxidative stress has been implicated in the pathogenesis of several neurodegenerative disorders, cancer, and aging. Oxidative stress occurs as a response to increased oxidants, decreased anti-oxidants, or failure to repair oxidative damage induced by reactive oxygen species (ROS). ROS are free radicals, reactive molecules containing oxygen, or molecules containing oxygen that generate free radicals. ROS include nitric oxide (NO), superoxide (O2-), peroxynitrite (ONOO—), and hydroxyl radical (OH—). In particular, the amino acid sequences according to the invention work as an H202 detoxifying enzyme, and possibly prevent lipid peroxidation and apoptosis. The amino acids of the present invention can metabolize and eliminate H2O2, partially avoiding the conversion of H2O2 to a hydroxyl radical. As such, the amino acids of the present invention work to protect against paraquat toxicity. PRDX5 is present in a broad range of intracellular locations, including mitochondria, peroxisomes, the cytosol, and to a lesser extent, the nucleus. By manipulating the targeting signals of the amino acid sequences, the amino acid sequences can be directed to specific subcellular localizations. For instance, the amino acid sequence may be directed to either or predominantly the mitochondria, peroxisomes, the cytosol, or the nucleus. Additionally, the amino acids of the present invention may also be able to quench extra cellular free radicals.

The present invention is also related to the nucleotide sequence encoding the amino acid sequence according to the invention and its regulatory sequences upstream said coding sequence. A nucleotide sequence encoding the polypeptide according to the invention is a genomic DNA (see SEQ ID NO: 10), a cDNA (see SEQ ID NOs: 1 or 20) or a mRNA, possibly comprising said upstream regulatory sequence. Advantageously, said nucleotide sequence presents more than 70%, advantageously more than 85%, more preferably more than 95% homology with SEQ ID NOs: 1 or 20, or its complementary strand.

According to a preferred embodiment of the present invention, said nucleotide sequence corresponds to the nucleotide sequence SEQ ID NOs: 1 or 20, its complementary strand or a portion thereof. “A portion of the nucleotide sequence SEQ ID NOs: 1 or 20” means any nucleotide sequence of more than 15 base pairs (such as a primer, a probe or an antisense nucleotide sequence) which allow the specific identification, reconstitution or blocking of the complete nucleotide sequence SEQ ID NOs: 1 or 20 (including its regulatory sequences upstream the coding sequence).

Said portions allow the specific identification, reconstitution or blocking by specific hybridization with the nucleotide sequence SEQ ID NOs: 1 or 20, preferably under standard stringent conditions, with sequences like probes or primers possibly labeled with a compound (radioactive compound, enzyme, fluorescent marker, etc.), and can be used in a specific diagnostic or dosage method like probe hybridization (see Sambrook et al., 9.47-9.51 in Molecular Cloning: A Laboratory Manual, Cold Spring Harbor, Laboratory Press, Cold Spring Harbor, New York (1989)), genetic amplification (like PCR (U.S. Pat. No. 4,683,195), LCR (Wu et al., Genomics 4, pp. 560-569), CPR (U.S. Pat. No. 5,011,769)).

Exemplary stringent hybridization conditions are as follows: hybridization at 42° C. in 50% formamide 5×SSC, 20 mM sodium phosphate, pH 6.8 washing in 0.2×SSC at 55° C. It is understood by those skilled in the art that variation of these conditions occur based on the length and GC nucleotide content of the sequence to be hybridized.

Formulas standard in the art are appropriated for determining exact hybridization conditions, see for instance Sambrook et al. (supra).

Preferred examples of said nucleotide sequence portions are as follows:

| | |
|---|---|
| Sequence | Position |

| | | |
|---|---|---|
| 5′-gccatcccagcagtggaggtgtttg-3′ | (SEQ ID | 217-241 |
| | NO 11) | |
| 5′-ttgaacagctctgccaggttcacc-3′ | (SEQ ID | 261-284 |
| | NO 12) | |
| 5′-tggaggtgtttgaaggggagccag-3′ | (SEQ ID | 230-253 |
| | NO 13) | |
| 5′-caggttcaccttgttccctggctc-3′ | (SEQ ID | 247-270 |
| | NO 14) | |
| 5′-gggtatgggactagctggcg-3′ | (SEQ ID | 33-52 |
| | NO 15) | |
| 5′-ctggccaacattccaattgcag-3′ | (SEQ ID | 747-768 |
| | NO 16) | |

and the sequences of, respectively, 601 (SEQ ID NO: 8), 604 (SEQ ID NO: 9) and 469 (SEQ ID NO: 7) base pairs corresponding to specific mRNA alternative splicing of the PRDX5 human nucleotide sequence as described in FIG. 4 (the known genomic sequence incorporating several introns and exons is represented in the sequence SEQ ID NO: 10).

Said sequences may be used for a genetic amplification or a probe hybridization as above-described.

The present invention is also related to a vector comprising the necessary elements for the injection, transfection or transduction of cells and having incorporated one or more of the nucleotide sequences according to the invention. The vector according to the invention is selected from the group consisting of viruses, plasmids, phagemides, cationic vesicles, liposomes or a mixture thereof. Said vector may comprise also one or more adjacent regulatory sequences (such as promoter(s), secretion and termination signal sequence (s)) advantageously operably linked to the nucleotide sequence according to the invention.

The present invention is also related to the cell transformed by said vector and expressing the polypeptide according to the invention.

The nucleotide sequence according to the invention can be also introduced in said cell by the formation of CaPO4-nucleic acid precipitate, DEAE-dextran nucleic acid complex or by electroporation.

Another aspect of the present invention is related to an inhibitor of the polypeptide according to the invention or the nucleotide sequence according to the invention (including the upstream sequences like promoter/operator regulatory sequence which may be inhibited by a cis- and/or transactivating repressor). Said inhibitor is advantageously an antibody or a fragment of said antibody such as an hyper variable portion of said antibody directed against the amino acid or nucleotide sequence of the polypeptide according to the invention. Other examples of inhibitors according to the invention are antisense nucleotide sequences which allow the blocking of the expression of the nucleotide sequence according to the invention.

In a preferred embodiment, the invention provides an antibody specifically recognizing the polypeptides of the present invention, or a specific epitope of said polypeptide. The term epitope refers to portions of a polypeptide having antigenic or immunogenic activity in an animal, preferably a mammal, and most preferably in a human. Epitope-bearing polypeptides of the present invention may be used to induce antibodies according to methods well known in the art including, but not limited to, in vivo immunization, in vitro immunization, phage display methods or ribosome display.

The antibody of the present invention relate to any polyclonal or monoclonal antibody binding to a protein of the present invention. The term “monoclonal antibody” used herein refers to an antibody composition having a homogeneous antibody population. The term is not limiting regarding the species or source of the antibody, nor is it intended to be limited by the manner in which it is made. Hence, the term “antibody” contemplates also antibodies derived from camels (Arabian and Bactrian), or the genus lama. Thus, the term “antibody” also refers to antibodies derived from phage display technology or drug screening programs. In addition, the term “antibody” also refers to humanized antibodies in which at least a portion of the framework regions of an immunoglobulin are derived from human immunoglobulin sequences and single chain antibodies as described in U.S. Pat. No. 4,946,778 and to fragments of antibodies such as Fab, F′(ab)2, Fv, and other fragments which retain the antigen binding function and specificity of the parent antibody. The term “antibody” also refers to diabodies, triabodies or multimeric (mono-, bi -, tetra- or polyvalent/ mono-, bi- or polyspecific) antibodies, as well as enzybodies, i.e. artificial antibodies with enzyme activity. Combinations of antibodies with any other molecule that increases affinity or specificity, are also contemplated within the term “antibody”. Antibodies also include modified forms (e.g. mPEGylated or polysialylated form (Fernandes & Gregoriadis 1997 Biochem. Biophys. Acta 1341:26-34) as well as covalently or non-covalently polymer bound forms. In addition, the term “antibody” also pertains to antibody-mimicking compounds of any nature, such as, for example, derived from lipids, carbohydrates, nucleic acids or analogues e.g. PNA, aptamers (see Jayasena, 1999 Clin. Chem. 45:1628-1650).

In specific embodiments, antibodies of the present invention cross-react with murine, goat, rat and/or rabbit homologues of human proteins and the corresponding epitopes thereof. Further included in the present invention are antibodies that bind polypeptides encoded by nucleic acids that hybridize to a polynucleotide of the present invention under stringent hybridization conditions (as described herein). As such, the present invention provides a method for detecting the polypeptides of the present invention, the method comprising the use of the antibodies in immunoassays for qualitatively or quantitatively measuring levels of the polypeptides of the present invention in biological samples.

Another aspect of the present invention is related to a diagnostic device (such as a diagnostic kit or a chromatographic column) comprising an element selected from the group consisting of the amino acid sequence of said polypeptide, its nucleotide sequence, and/or the inhibitor according to the invention or a fragment thereof as above-described. Said diagnostic device may comprise also necessary reactants and media for the diagnostic and/or dosage of the nucleotide and/or amino acid sequence of the polypeptide according to the invention, which are based upon the method selected from the group consisting of in situ hybridization, hybridization by labeled antibodies, especially RIA (Radio Immuno Assay) or ELISA (Enzymes Linked Immuno-Sorbent Assay) technologies, detection upon filter, upon solid support, in solution, in sandwich, upon gel, dot blot hybridization, Northern blot hybridization, Southern blot hybridization, isotopic or non-isotopic labeling (by immunofluorescence or biotinilised probes), genetic amplification, (especially by PCR or LCR), double immunodiffusion technique, counter-electrophoresis technique, haemagglutination or a mixture thereof.

Another aspect of the present invention concerns a diagnosis method wherein a biological sample from the patient, such as cephalo-rachidian fluid, serum, blood, plasma, urine, broncho-alveolar lavage, stomach lavage, etc., is isolated from the patient, and is put in contact with the diagnostic device according to the invention for the diagnosis or the monitoring of an injury or a disease, preferably a lung injury or an oxidative stress-related disorder, affected by the presence of pro-oxidant agent or oxidative stress such as specific cardiovascular diseases like arteriosclerosis, neurodegenerative disorders (Alzheimer's disease, Parkinson's disease, amyotrophic lateral sclerosis), apoptosis, inflammatory reactions, allergic reactions such as asthma, hay fever and eczema, high bone mass syndrome, osteopetrosis, osteoporosis-pseudoglioma syndrome, and Bardet-Biedl syndrome 1. Said diagnosis and monitoring upon one or more biological samples obtained from several tissues from the patient can be advantageously obtained by one or more of the methods above-described, which could be adapted according to the specific biological sample by the person skilled in the art.

Therefore, the product according to the invention could be used as a marker for the above identified injuries, diseases or disorders in a broad spectrum of tissues as shown in the enclosed FIG. 1.

A further aspect of the present invention is related to a pharmaceutical composition comprising a pharmaceutically acceptable carrier and an element selected from the group consisting of the nucleotide sequence, the amino acid sequence of the polypeptide according to the invention, the inhibitor directed against said sequences and/or one or more portions thereof.

Another aspect of the present invention is related to the use of the pharmaceutical composition according to the invention for the manufacture of a medicament in the treatment and/or the prevention of lung injuries and/or diseases or of oxidative stress-related disorders.

The present invention relates also to a prevention and/or treatment method of a patient, especially a human patient, preferably affected by lung injuries and/or diseases or by oxidative stress-related disorders, wherein a sufficient amount of the pharmaceutical composition according to the invention is administered to said patient in order to treat, avoid and/or reduce the symptoms of said injuries and/or diseases.

Other injuries and/or diseases which can be prevented and/or treated are injuries and/or diseases affected by the presence of pro-oxidant agents or oxidative stress, such as specific cardio-vascular diseases like arteriosclerosis, neurodegenerative disorders such as Alzheimer's disease, Parkinson's disease, amyotrophic lateral sclerosis, apoptosis and inflammatory reactions and some allergic reactions such as asthma, hay fever and eczema, high bone mass syndrome, osteopetrosis, osteoporosis-pseudoglioma syndrome, and Bardet-Biedl syndrome 1.

Accordingly, the present invention relates also to a pharmaceutical composition in orally administrable dosage form, comprising:
(a) the amino acid sequence having more than 70% homology with the sequence SEQ ID NO 1 or 21, or a pharmaceutically acceptable salt or derivative thereof, and
(b) possibly a pharmaceutically acceptable reductant and/or electron donor.

For instance, a pharmaceutically acceptable reductant and/or electron donor may be dithiotreitol (DTT). The pharmaceutically acceptable reductant and/or electron donor may display a synergistic neuroprotective effect on neuronal cell death.

The amino acid sequences of the present invention provide neuroprotection against excitotoxic stress. Excitotoxic stress relates to possessing the property of exciting and then poisoning cells or tissues; examples include nerve injury produced by glutamate. In particular, the amino acid sequences of the present invention provide a neuroprotective effect against injuries to the CNS, such as, for instance, brain lesions, neuronal cell death, periventricular leukomalacie. Moreover, PRDX5 may prevent and/or ameliorate the neurological handicaps of perinatal origin, as well as increase the survival rate of preterm infants. PRDX5 may also be protective in other neurodegenerative conditions associated with oxidative stress, especially if related to NMDA receptor-mediated excitotoxity.

Accordingly, the present invention relates also to a method of treating neurotoxic injury in a patient suffering of said injury by administering to said patient a composition comprising the amino acid sequence having more than 70% homology with the sequence SEQ ID NOs: 1 or 21, its pharmaceutically acceptable salts or derivatives and pharmaceutically acceptable esters, and a pharmaceutically acceptable carrier, wherein said compound is present in said composition in an amount effective to treat said neurotoxic injury.

Neurotoxic injury is caused by a neurotoxin, which is any substance which possesses the ability to damage or destroy nerve tissue. The action of neurotoxins may be direct by injuring or destroying neurotransmitters or the cells themselves which produce them, or indirectly by initiating an adverse secondary body response (i.e. immunologic) thus accomplishing the same result. In this regard it is connoted that the neurotoxins may induce and/or sustain oxidative stress in neuronal cells.

The present invention relates also to a method of decreasing the effect of excitotoxic injury in a patient, having said injury, comprising administrating to said patient a composition comprising the amino acid sequence having more than 70% homology with the sequence SEQ ID NOs: 1 or 21, its pharmaceutically acceptable salts or derivatives and pharmaceutically acceptable esters, and a pharmaceutically acceptable carrier, wherein said compound is present in said composition in an amount effective to treat said excitotoxic injury in said patient.

The present invention relates also to a method as described herein, wherein said excitotoxic injury is caused by oxidative stress.

The present invention relates also to a method as described herein, wherein said excitotoxic injury affects neuronal cells.

In addition to the above mentioned protection, the amino acid sequences of the present invention also play a protective role against oxidative stress in human cartilage. In particular, the amino acid sequences of the present invention may act as a protection to cartilage, for instance in chondrocytes, against ROS-induced oxidative damage. Hence, these amino acids may have a therapeutic value in the prevention and treatment of OA.

Accordingly, the present invention relates also to a method as described herein, wherein said excitotoxic injury is osteoarthritis.

It will be understood by the skilled artisan that the present invention relates also methods wherein the endogenous production of PRDX5 is increased in patients suffering from any of the above-mentioned diseases or conditions. The endogenous production may be increased, for instance, by removing a translational block for the synthesis of PRDX5.

The pharmaceutically acceptable carrier according to the invention is any compatible non-toxic substance suitable for administering the composition according to the invention to a human patient.

The pharmaceutical composition according to the invention may be prepared by the methods, generally applied by the person skilled in the art in the preparation of various pharmaceutical compositions, wherein the percentage of the active compound/pharmaceutically acceptable carrier can vary within very large ranges, only limited by the tolerance of the patient to said pharmaceutical composition, and wherein the limits are particularly determined by the frequency of administration and the possible side-effects of the active compounds or its pharmaceutically acceptable carrier. Thus, the present invention relates to pharmaceutical preparations which as active constituents contain an effective dose of at least one of the amino acids as described herein, or an active part thereof in addition to customary pharmaceutically innocuous excipients and auxiliaries. The pharmaceutical preparations normally contain 0.1 to 90% by weight of a compound of formula (I). The pharmaceutical preparations can be prepared in a manner known per se to one of skill in the art. For this purpose, at least one of the amino acids as described herein, or an active part thereof, together with one or more solid or liquid pharmaceutical excipients and/or auxiliaries and, if desired, in combination with other pharmaceutical active compounds, are brought into a suitable administration form or dosage form which can then be used as a pharmaceutical in human medicine or veterinary medicine.

Pharmaceuticals which contain a compound according to the invention can be administered orally, parenterally, e.g., intravenously, rectally, by inhalation, or topically, the preferred administration being dependent on the individual case, e.g., the particular course of the disorder to be treated. Oral administration is preferred. Pharmaceutically acceptable carriers according to the invention suitable for oral administration are the ones well known by the person skilled in the art, such as tablets, coated or non-coated pills, capsules, spray-gas, patches, gels, solutions or syrups. Nonetheless, pharmaceutically acceptable carriers vary according to the mode of administration (intravenous, intramuscular, subcutaneous, parenteral, etc.), and may comprise also adjuvants well known by the person skilled in the art to increase, reduce and/or regulate humoral, local and/or cellular response of the immune system.

The person skilled in the art is familiar on the basis of his expert knowledge with the auxiliaries which are suitable for the desired pharmaceutical formulation. Beside solvents, gel-forming agents, suppository bases, tablet auxiliaries and other active compound carriers, antioxidants, dispersants, emulsifiers, antifoams, flavor corrigents, preservatives, solubilizers, agents for achieving a depot effect, buffer substances or colorants are also useful.

Another aspect of the invention is related to the use of the diagnostic device according to the invention for performing upon the patient or upon a biological fluid obtained from the patient, a diagnosis, a dosage and/or a monitoring of the above-mentioned injuries or diseases or oxidative stress-related disorders affecting the patient.

A further aspect of the present invention is related to a cell or a non-human animal, preferably a mammal such as a mouse or a rat, transformed by the vector according to the invention and over expressing the polypeptide according to the invention, or a non-human animal, preferably a mammal such as a mouse or a rat, genetically modified by a partial or total deletion of its genomic sequence encoding the polypeptide according to the invention (knock-out non-human mammal) and obtained by methods well known by the person skilled in the art such as the one described by Kahn et al. (Cell, Vol. 92, pp.593596 (March 1998)).

Other examples of genetically modified nonhuman animals according to the invention may be a transgenic non-human animal comprising an inhibitor according to the invention, preferably an antisense nucleic acid sequence complementary to the nucleotide sequence according to the invention so placed as to be transcribed into antisense mRNA which is complementary to the nucleotide sequence according to the invention and which hybridizes to said nucleotide sequence, thereby reducing or blocking its translation.

Further aspects of the present invention will be described in the enclosed non-limiting examples in reference to the following Figures.

All the references cited above or below are explicitly incorporated herein by reference.

### BRIEF DESCRIPTION OF THE DRAWINGS

FIG. 1 Dot blot analysis of mRNA encoding the polypeptide according to the invention (PRDX5 denoted as B18) in various types of human tissues.

FIG. 2 Northern blot analysis of mRNA encoding the polypeptide according to the invention (PRDX5 denoted as B18) in a rat lung after administration of lipopolysaccharides (LPS) inducing an inflammatory reaction of the lung.

FIG. 3 Northern blot analysis of mRNA encoding the polypeptide according to the invention (PRDX5 denoted as B18) in a rat lung after intraperitoneal injection of pneumotoxicants.

FIG. 4 Schematic representation of the human genomic sequence, the complete cDNA sequence and the corresponding amino acid sequence.

FIG. 5 Alignment of the sequences of the human PRDX5 (B18) polypeptide according to the invention with the corresponding rat and mouse sequences, respectively. B18hum: human PRDX5; B18rat: rat PRDX5; B18mouse: mouse PRDX5.

FIG. 6 Expression of PRDX5 protein in human articular cartilage. A: PRDX5 protein bands (upper panel) and actin bands (lower panel) detected by Western blotting in normal (N) and osteoarthritic cartilage (OA). B: Comparison of PRDX5 protein levels in normal and osteoarthritic cartilage (n=4) by densitometry analysis. PRDX5 protein level was expressed by the ratio of the net intensity of the PRDX5 band to the actin band of the same sample. Bars represent mean±SEM. *p<0.05 OA vs normal.

FIG. 7 Effects of TNFα and IL-1β on PRDX5 protein expression in human articular cartilage explant culture. Cartilage explants from OA patients were precultured with TNFα (100 ng/ml) (panel A) or IL-1β (10 ng/ml) (panel B) for the indicated times, and subject to Western blot analysis using anti-PRDX5 and anti-actin antibodies as described in Section 9.3.

FIG. 8 Effects of TNFα on PRDX5 mRNA and protein expression in cultured human articular cartilage chondrocytes. Chondrocytes isolated from OA cartilage were precultured with TNFα (100 ng/ml) for the indicated times. A: PRDX5 mRNA was detected by Northern blot analysis. B: PRDX5 protein expression was analyzed by Western blot.

FIG. 9 Effect of catalase and/or TNFα on chondrocyte H2O2 production and PRDX5 protein expression. A: Flow cytometric analysis of intracellular H2O2 production by chondrocytes treated with TNFα (100 ng/ml) or TNFα +catalase (500 u/ml) for the indicated times. Catalase was added to culture 2 hours before the addition of TNFα. B: Western blot analysis of PRDX5 protein from chondrocytes treated as above. Results were representatives of three separate experiments using chondrocytes isolated from three OA cartilages.

FIG. 10 PRDX5 protects the newborn mouse brain against ibotenate-induced lesions. Cresyl violet-stained sections showing brain lesions induced by ibotenate injected at P5 and studied at the age of P10. A. Brain from pup co-treated with intracerebral ibotenate and intraperitoneal PBS, showing the typical neuronal loss in layers II-VI (arrow) and the white matter cystic lesion (CL). B. Brain from pup co-treated with intracerebral ibotenate and intraperitoneal 10 mg/kg PRDX5. LV, lateral ventricle. Bar: 40 μm.

FIG. 11A. Effects of different doses of PRDX5 (PRDX) administered immediately after ibotenate on excitotoxic brain lesions. B. Effects of delayed (time elapsed between ibotenate and PRDX5 injections is indicated on the X-axis) administration of 10 mg/kg PRDX5 (PRDX) on ibotenate-induced lesions. C. Effects of different doses of catalase (Cat) administered immediately after ibotenate on excitotoxic brain lesions. D. Effects of different doses of catalase-PEG (cat-PEG in U/kg) or of PEG (in mg/kg) administered immediately after ibotenate on excitotoxic brain lesions E. Effects of different doses of N-acetylcysteine (NAC) administered immediately after ibotenate on excitotoxic brain lesions. F. Effects of 10 mg/kg PRDX5 (PRDX), 250 mg/kg N-acetylcysteine (NAC) or 600,000 U/kg catalase-PEG (Cat-PEG) administered immediately after S-bromowillardiine on excitotoxic brain lesions. In all experiments, lesions were produced on P5 and analyzed on P10. Bars represent mean length of the lesions in the fronto-occipital axis +SEM. Asterisks indicate difference from control (black bar); * p<0.05, ** p<0.01, *** p<0.001 in ANOVA with Dunnet's multiple comparison test (A, D-F) or in contrasts (B). Numbers in brackets are the numbers of animals used in each experimental group.

FIG. 12 PRDX5 prevents ibotenate-induced neural cell death. A-B. Tunel stained sections showing cell death induced by ibotenate +PBS (A) or ibotenate 10 mg/kg PRDX5 (B) injected on P5 and studied on P6. PRDX, PRDX5; CL, cystic lesion in the white matter. Bar: 50 μm. C. Quantitative analysis of Tunel-stained nuclei in the cortical plate and white matter lesions produced by ibotenate injection on P5 and studied 8 or 48 hours later. Bars represent means [symbol] SEM. Asterisks indicate difference from PBS group (* p<0.05 in Student's t-test).

FIG. 13 Effect of PRDX5 (PRDX) and dithiothreitol (DTT) on NMDA-induced (300 μM) in vitro neuronal cell death. Quantitative analysis of cells showing apoptotic features (pycnotic nucleus) after staining with Bis-benzimide (Hoechst 33258). Number of pycnotic nuclei was divided by the total cell number and, for each experimental group, this ratio was normalized to ratio obtained in controls. Results are shown as means +SEM. Asterisks indicate statistically significant differences between controls (PBS group) and experimental groups (* p<0.05, ** p<0.01, *** p<0.001 in ANOVA with Dunnett's multiple comparison test).

FIG. 14 Exogenous recombinant PRDX5 is detected in cultured neurons. Immunodetection of PRDX5 in neuronal cultures. Immunoreactivity for PRDX5 in neuronal cell cultures incubated during 30 min with 100 μM recombinant PRDX5 (A-1) or phosphate buffer saline (B-1). In (A-1); a cell showing a typical neuronal phenotype and incubated with recombinant PRDX5 is intensely labeled (arrow). (A-2) and (B-2) DAPI staining for nuclear visualization of cells immunostained in (A-1) and (B-1) respectively. Bar: 20 μm.

Table 1 Homologies of the PRDX5 (B18) proteins (162 amino acid) with other proteins

Table 2 Homologies of the human PRDX5 cDNA (805 nucleotides) with GenBank, EMBL, DDBJ and PDB deposited nucleotide sequences

### EXAMPLE 1

### Homology Between the PRDX5 Polypeptide According to the Invention With Other Known Nucleotide or Amino Acid Sequences

The BLAST 2.0 software (gapped BLAST at the NCBI Internet site) was used for searching for homologies between human PRDX5 (162 amino acids) and known polypeptides in databases (GenBank, SwissProt). Said search did not give perfect alignment with known peptides from different species (Table 1). Homologies of the human PRDX5 cDNA (805 nucleotides) with GenBank, EMBL, DDBJ and PDB deposited nucleotide sequences (Table 2) and GenBank Expression Sequence TAGS (ESTs) were noted.

In the Table 2, an identity of 98% has been obtained with the alignment of 259 nucleotides of cDNA PRDX5, which comprises in its totality 805 nucleotides, with 263 nucleotides of U82616 cDNA. A similar identity has been obtained with the U82615 sequence.

The sequence SEQ ID NO: 1 comprising 805 nucleotides presents a homology with several EST sequences obtained from a human and from a mouse, having the following references:

Human:

AA130751, N42215, W38597, N91311, N68467, AA187737, N68916, W00593, R88950, AA181884, H20154, H66666

Mouse:

AA220019, AA123351, AA087129, AA255021, AA249897, W71344

### EXAMPLE 2

### Tissue Detection

A human RNA master Blot (Clontech) containing 100-500 ng of poly-A+human RNA in each dot (normalized to the mRNA expression levels of eight different housekeeping genes) was hybridized with a 554 bp-long PRDX5 probe labeled with 32P, and quantified using Phosphorimaging Technology.

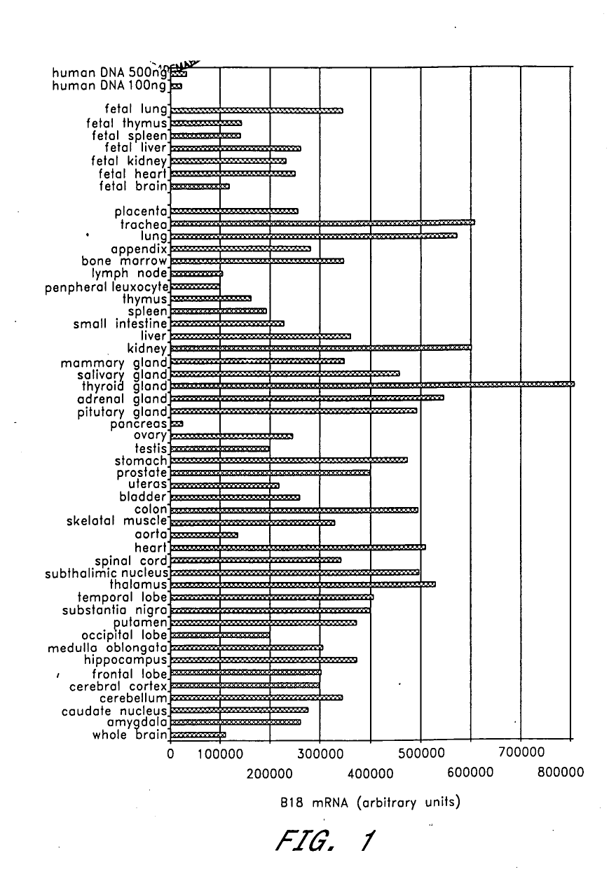

As shown in FIG. 1, PRDX5 mRNA is present in all tissues examined but predominantly in trachea, lung, kidney, thyroid gland, stomach, colon, heart and some regions of the brain. Highest expression has been noted in the thyroid tissue. This presence is probably correlated with the possible antioxidant activity of the PRDX5 polypeptide according to the invention.

### Example 3

### Inflammatory Reaction

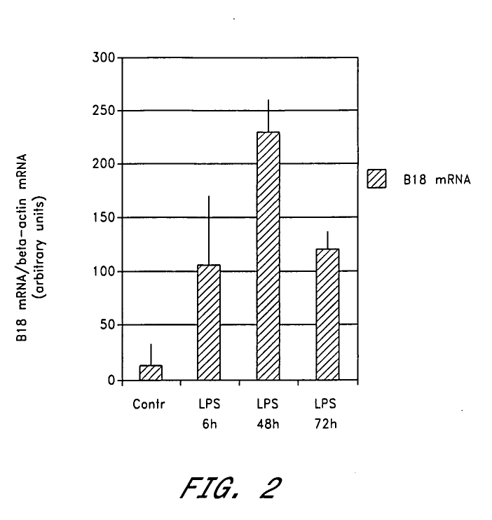

FIG. 2 represents a Northern blot analysis of rat lung mRNA after 6, 48 and 72 hours after lipopolysaccharides (LPS) instillation inducing an inflammatory reaction in the lung.

A Northern blot containing 15 μg of total RNA in each lane was hybridized with a 225 bp-long rat PRDX5 probe, stripped and reprobed with a 572 bp-long rat β-actin probe, both labeled with 32P. Northern blot was quantified using Phosphorimaging Technology and the PRDX5 mRNA data were normalized to β-actin mRNA level.

### EXAMPLE 4

### Pneumotoxic Reaction

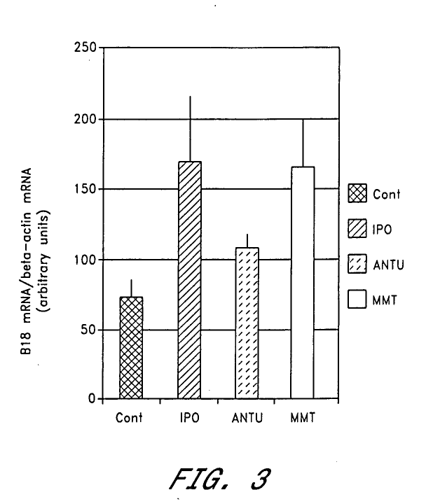

FIG. 3 represents a Northern blot analysis of rat lung mRNA after intraperitoneal injection of pneumotoxicants (4-ipomeanol,1-(3-furyl)-4-hydroxypentanone (IPO), methyl-cyclopentadienyl manganese tricarbonyl (MMT) or alpha naphtylthiourea (ANTU)). These agents are known to induce in the lung acute lesions of Clara (IPO) and alveolar cells (MMT) as well as increasing the permeability of the alveolar/blood barrier (ANTU). A Northern blot containing 15 μg of total RNA in each lane was hybridized with a 225 bp-long rat PRDX5 probe, stripped and reprobed with a 572 bp-long β-actin probe both labeled with 32P.

The Northern blot was quantified using Phosphorimaging Technology and rat PRDX5 mRNA data were normalized to p-actin mRNA level.

### Example 5

### Protein Activity of the PRDX5 Polypeptide

An amino analysis of the complete human PRDX5 amino acid sequence shows that said polypeptide presents specific portions showing an homology with other antioxidant enzymes (starting from a Leucine at position 36 until a Cysteine at position 47) and an other portion having an important homology with beta chains of ATP synthase (starting from a Glutamic acid at position 13 until a Glycine in position 38).

Furthermore, the PRDX5 amino acid sequence according to the invention shows an important homology with an Aspergillus fumigatus allergen (34% identity and 60% homology by using clustal V sequence alignment), especially upon the portion of said PRDX5 polypeptide having possible antioxidant properties. Therefore, it is possible that a peroxisomal protein (possibly homologous to PRDX5 protein) is able to induce and to bind IgE from patients sensitized to Aspergillus fumigatus peroxisomal proteins after an induction of the patient immune system with Aspergillus fumigatus allergen. This mechanism can be compared to a reaction obtained with the manganese superoxide dismutase (MnSOD) wherein the human MnSOD is able to bind to IgE from patients sensitized to Aspergillus fumigatus MnSOD.

Furthermore, the Inventors have identified a portion of the PRDX5 human polypeptide which presents an homology with a Cyclophilin-binding domain of Candida boidinii PMP20 (preceptor1 of the immuno-suppressant drug cyclosporine A). Said possible Cyclophilin-binding domain is starting from the Threonine in position 150 until the Leucine in position 161.

### EXAMPLE 6

### PRDX5 Human Gene and mRNA Alternative Splicing

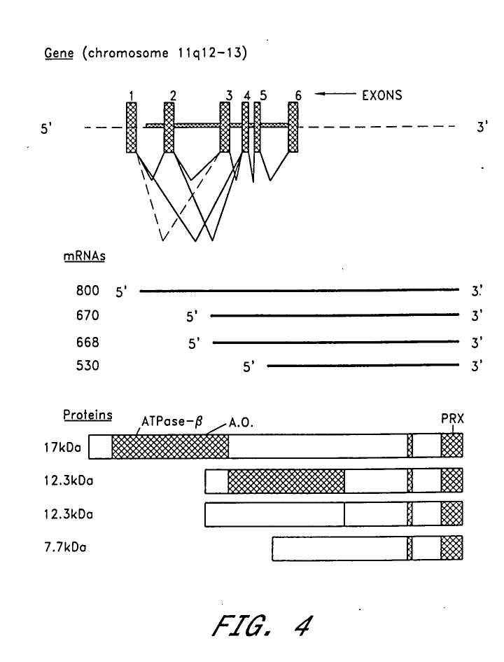

As represented in the enclosed FIG. 4, the Inventors have identified upon the genomic DNA (SEQ ID NO: 10) 5 exons and 5 introns. By RT-PCR (using primers 5′-gggtatgggactagctggcg-3′ and 5′-ctggccaacattccaattgcag-3′) and according to the genomic sequence, 4 different cDNAs corresponding to the transcription of the said genomic DNA have been identified in human lung and in human brain. A first cDNA of 736 bp corresponds to the cDNA encoding the complete amino acid sequence of the PRDX5 protein according to the invention. However, 3 other cDNAs of 601, 604 and 469 bp were also identified, and comprise specific splicings of one or more exons.

Therefore, another aspect of the present invention is related to said specific portions of the complete genomic or cDNA nucleotide sequence according to the invention or to specific portions of the complete amino acid sequence of the PRDX5 protein according to the invention, which could be used also as specific markers of the PRDX5 activity, preferably the anti-oxidative activity.

### EXAMPLE 7

### Knock-Out Mouse

Exons of a mouse genomic sequence encoding the PRDX5 polypeptide according to the invention have been deleted by homologous recombination. Said homologous recombination has been obtained with a genetic sequence comprising a neomycin resistant gene. The targeting vector with said gene and a thymidine kinase (in order to eliminate non-homologous recombinants with ganciclovir) has been prepared. Said recombination was used for the deletion of one or more exons of the PRDX5 polypeptide. After electroporation of ES cells with the targeting vector, positive clones having incorporated homologous recombination were identified by Southern blot with labeled probes. Aggregation of said positive clones with a morula from a Swiss pseudo-pregnant mouse produces several chimeric mice which survive after birth. Several homozygote mice are obtained by cross-breeding and are used as a model for the above-mentioned diseases.

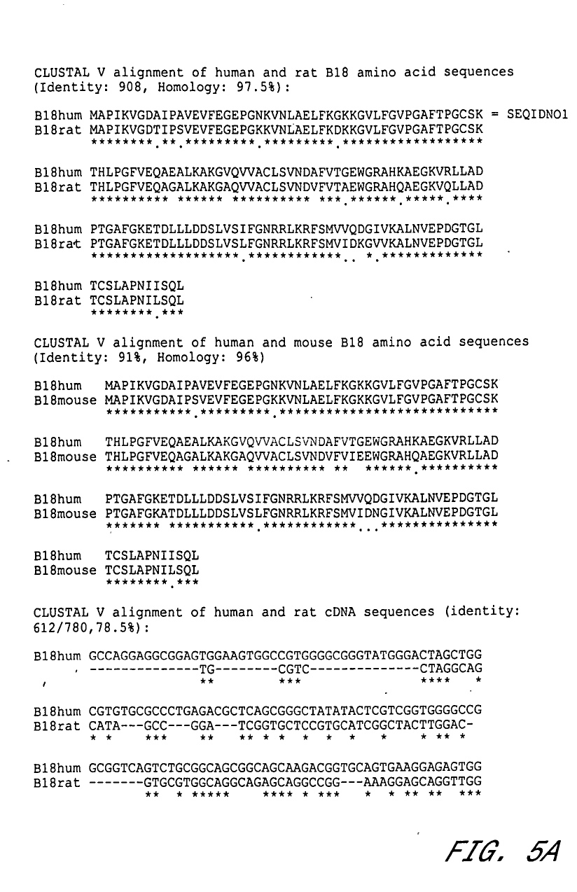

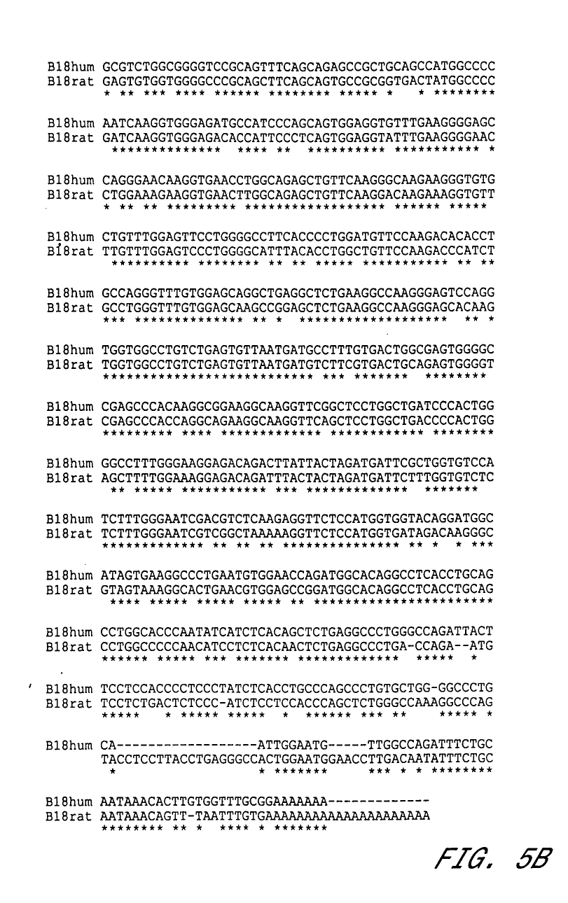

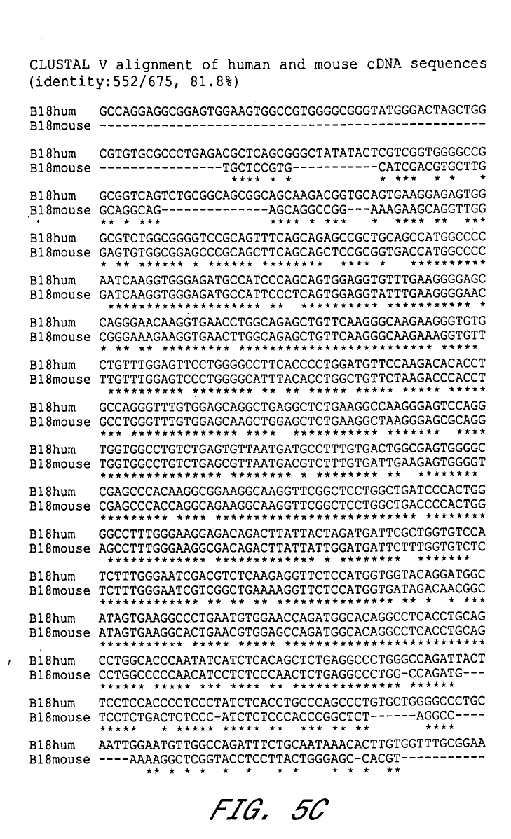

Similar experiments may be done with another mammal whose PRDX5 sequence is known (the PRDX5 sequence of a mouse and a rat and their alignment with the human sequence is shown in the enclosed FIG. 5).

### EXAMPLE 8

### Chromosome Localization of Human PRDX5 Gene

Radiation hybrid clones (GeneBridge 4 Radiation Hybrid Panel, Research Genetics) were used for performing chromosome localization by PCR with two different pairs of primers

| | |
|---|---|
| (5′-caggttcaccttgttccctggctc-3t; | (SEQ ID NO: 14) |
| 5′-atgttatgcaaccctttgcgacac-3′; | (SEQ ID NO: 17) |
| and | |
| 5′-gtgtttgaaggggagccagggaac-3′; | (SEQ ID NO: 18) |
| 5′-agagacagggtttcaccatcttgg-3′. | (SEQ ID NO: 19)) |

The Inventors have located PRDX5 genomic sequence on human chromosome 11q13. PRDX5 gene has been located 7.15-6.1 cR from marker D11S913 between markers D11S1963 and D11S4407 (Genome Database internet site).

Unknown genes linked to different disorders have been localised in the same region of chromosome 11. Therefore, the PRDX5 gene is possibly associated with these disorders:
atopy (atopic hypersensitivity: asthma, hay fever, and eczema; MIM No 147050 at OMIM of NCBI internet site),
high bone mass syndrome (MIM No 601884),
osteopetrosis (MIM No 259700),
osteoporosis-pseudoglioma syndrome (MIM No 259770) and
Bardet-Biedl syndrome 1 (MIM No 209901).

### Example 9

### Expression and Regulation of PRDX5 in Human Osteoarthritis

9.1 Tissue Collection and Culture

Human tissue collection in this study was approved by the South Eastern Sydney Area Health Service Ethics Committee, Australia. Human osteoarthritic cartilage (n=4) was removed from knee joints of patients with osteoarthritis (OA) undergoing total knee-replacement surgery. Significant osteoarthritis was identified by preoperative radiography. Normal human cartilage (n=4) was removed immediately adjacent to the insertion site of supraspinatus tendon as part of the surgical procedure to reattach the tendon to bone. Significant osteoarthritis was excluded by plain anterior-posterior (AP) and lateral radiographs of the shoulder, and by arthroscopic examination of the glenohumeral joint prior open rotator culf repair.

For RNA and protein extraction, cartilage tissues were snap frozen in liquid nitrogen upon collection and stored at −800 C until RNA extraction and tissue homogenization were performed. For tissue culture, cartilage explant discs were made using a 4 mm diameter disposable biopsy punch (SMS Inc, Columbia, Md.) with an average thickness of 2 mm. Three discs were placed in each well of 24 well plates (1 ml final volume). Chondrocytes were isolated from cartilage by collagenase digestion (Jang et al. 1999 Arthritis Rheum. 42:2410-2417) and plated in 25 cm2 flasks (5×105) containing Dulbecco's Modified Essential Medium (DMEM) supplemented with 10% fetal bovine serum (FCS). Primary chondrocyte monolayer cultures and cartilage explants were incubated at 37° C. in a 5% CO2 humidified atmosphere. Culture medium was replaced with serum-free DMEM for all experiments.

To mimic in vivo pathological conditions, tumor necrosing factor α (TNFα) 100 ng/ml, and interleukin 1 β (IL-1β) 10 ng/ml (R&D System, Minneapolis, Minn.) were added to explant and chondrocyte cultures. Catalase (500 u/ml) (Sigma, St. Louis, Mo.) was used as an H2O2 scavenger and added to chondrocyte culture 2 hours before TNFα stimulation. Tissue and cells were harvested at 3, 6, 12, 24 and 48 hours after the addition of TNFα or IL-1β for total RNA extraction, protein preparation and intracellular H2O2 determination.

9.2 Northern Blotting

Total RNA was isolated from human chondrocytes using Trizol reagent (Life Technologies, Melbourne, Australia) following the manufacturer's instructions. Denatured RNA samples (20 μg) were fractionated by electrophoresis in a denaturing 1% (w/v) agarose gel, transferred to a GeneScreen Plus nylon membrane (NEN Life Science Products, Boston, Mass.), cross-linked using an ultraviolet cross-linker (Ultra-Lum, Carson, Calif.), and hybridized with 32P-labeled human PRDX5 cDNA probe. The blots were subsequently stripped and reprobed with 32P-labeled human β-actin cDNA. All probes were radiolabeled by random priming (Promega, Sydney, Australia).

9.3 Western Blotting

Human cartilage tissue was ground to a fine powder in liquid nitrogen using a mortar and pestle, and homogenized in 5 volumes of Tris-HCL buffer (pH 7.4) containing a protease inhibitor cocktail (2 μg/ml leupeptin, 5 μg/ml pepstatin A, 2 mg/ml of EDTA-Na2, 500 μg/ml AEBSF and 1 μg/ml E-64) (Sigma, St. Louis, Mo.). Chondrocyte lysate was prepared by sonication in the same homogenization buffer. Proteins in the tissue homogenates or cell lysate were denatured by boiling for 5 minutes in 2% (w/v) SDS sample buffer and fractionated by electrophoresis on 12.5% (w/v) SDS-polyacrylamide gel. The fractionated proteins were electrophoretically transferred to a PVDF membrane, and the blots were blocked with 5% (w/v) non-fat dry milk TTBS solution containing 25 mM Tris, pH 7.5, 150 mM NaCl, 0.05% (w/v) Tween 20. After washing in TTBS, the blots were incubated for one hour with polyclonal anti-PRDX5 antibody [19] at 1:2000 dilution, followed by horseradish peroxidase-conjugated anti-rabbit secondary antibodies (Chemicon, Melbourne, Australia). Immunoreactive bands were detected by ECL reagents (Pierce, Rockford, Ill.). The membranes were stripped with Restore Western Blot Stripping Buffer (Pierce, Rockford, Ill.) and reprobed using a rabbit anti-human actin antibody (Sigma, St. Louis, Mo.) as a house-keeping control. The net intensity of the PRDX5 band was analyzed by Bio-Rad Quantity One image quantification system (Bio-Rad, Hercules, CA). PRDX5 protein level was expressed by the ratio of the net intensity of the PRDX5 band to actin band of the same sample.

9.4 Intracellular H2O2 Assay

Intracellular H2O2 generation was assessed in chondrocytes by a flow cytometric technique based on the methods of Sattler et al. (1999, Blood 93:2928-2935), in which 2′,7′-dichloro-fluorescin-diacetate (DCF-DA) (Sigma, St. Louis, Mo.) was used. DCF-DA is a cell permeable dye commonly used to monitor intracellular changes in ROS (more specifically for H202). DCF-DA readily diffuses into cells where it is hydrolyzed to the non-fluorescent derivative 2′,7′-dichloro-fluorescin (DFC) and is trapped within the cells. DCF becomes highly fluorescent when oxidized by either H2O2 or superoxide. Cellular fluorescence intensity is directly proportional to the level of intracellular H202 produced by the cells and can be monitored by flow cytometry. Chondrocytes were freed from culture flasks by trypsin-EDTA. Cells (106) were incubated with 5 μM of DCF-DA for 5 minutes at 37 oC and subsequently washed twice in cold PBS before analysis by a flow cytometer (FACSort, Becton Dickinson System, San Jose, Calif.) with Cell Quest Software (Becton Dickinson). The fluorescence of oxidized DCF was measured with an excitation wavelength of 488 nm and emission wavelength of 525 nm. Mean fluorescence intensity (MFI) for 10,000 cells was recorded for each sample. Fluorescence relative variance (FRV) was calculated for each sample (S) compared with control (C) (loaded cells without TNFα treatment): FRV=[(MFI(S)−MFI(C) )/MFI(C)

9.5 Statistical Analysis

Comparison of PRDX5 expression in normal and osteoarthritic cartilage was made by Mann-Whitney Rank Sum Test using SigmaStat (Jandel Scientific, San Rafael, Calif.). A p value <0.05 was considered statistically significant.

Results

9.6 PRDX5 Protein is Constitutively Expressed in Human Cartilage and Upregulated in Osteoarthritic Cartilage

A rabbit antibody was raised against recombinant human PRDX5 (Wang et al. 2001, Biochem. Biophys. Res. Commun. 284:667-673). With this antibody, PRDX5 protein expression was readily detected by Western blot of cartilage tissue homogenates (FIG. 6). To semi-quantify the PRDX5 protein expression in both normal and osteoarthritic cartilage, all blots were reprobed with an anti-actin antibody and the results were expressed as a density ratio of PRDX5:actin. It was observed that osteoarthritic cartilage had more fibrin tissue and less cellularity than normal cartilage. Even if the same amount of total protein were loaded for Western blotting, one would expect less cellular protein in osteoarthritic cartilage than that in normal cartilage. Therefore, the normalization of PRDX5 protein with actin is important for the semi-quantification. FIG. 6 shows a significantly higher PRDX5 protein level in osteoarthritic cartilage than that in normal cartilage, suggesting an upregulation of PRDX5 in osteoarthritis.

9.7 PRDX5 Expression is Regulated by Inflammatory Cytokines

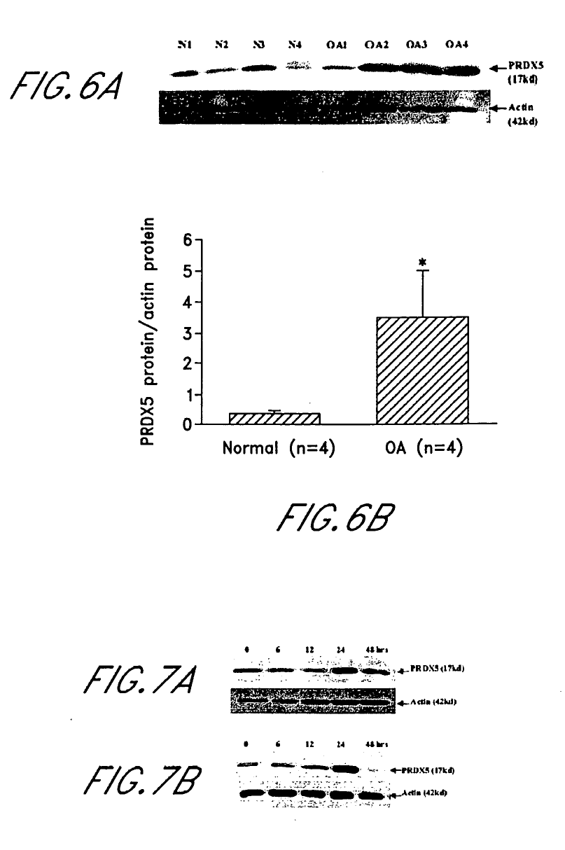

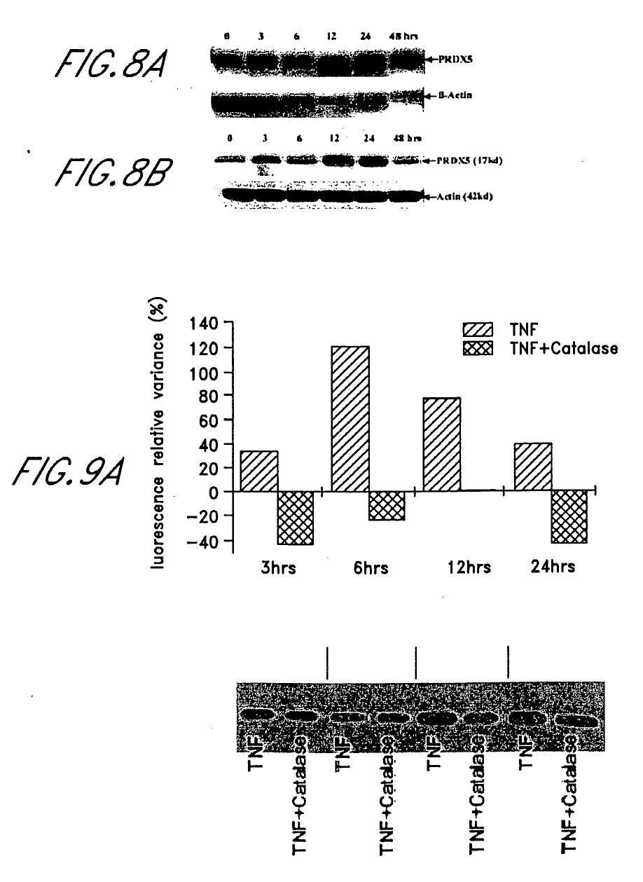

To explore the mechanisms by which the PRDX5 expression in human chondrocytes is regulated, we introduce inflammatory cytokines to the cartilage explant culture and primary chondrocyte culture, as cytokines have been shown to be implicated in osteoarthritis. As shown in FIG. 7, TNFα and IL-1β upregulated PRDX5 protein expression in the cartilage explant culture 24 hours after the cytokine challenge. At 48 hours, the PRDX5 protein levels decreased to near baseline in TNFα treated cartilage and below baseline in IL-1β treated cartilage. A similar phenomenon was observed in primary chondrocyte culture (FIG. 8). Both PRDX5 mRNA and protein expression started to increase 3 hours after TNFα stimulation, and reached their peak levels at 12 and 24 hours. The protein level returned to baseline at 48 hours while mRNA level remained high.

9.8 H2O2 Might be an Important Mediator for the PRDX5 Upregulation in Human Chondrocytes

Cytokines can induce intracellular H202 production in chondrocytes. We hypothesized that the increased PRDX5 expression in stimulated chondrocytes was a cellular response to intracellular flux of H2O2. To test this hypothesis, intracellular H2O2 induction was measured using a flow cytometric technique. Catalase was introduced to the chondrocyte culture system to test if scavenging H2O2 would affect the PRDX5 expression level. As shown in FIG. 9A, the intracellular H2O2 level in cultured human chondrocytes started to rise 3 hours after a TNFα challenge, reached its peak at 6 hours, and started to decline at 12 hours when the PRDX5 protein expression reached its peak (FIG. 9B). The addition of catalase to the chondrocyte culture prior to exposure to TNFα significantly inhibited TNFα-induced intracellular H2O2 production (FIG. 9A), as well as the enhanced PRDX5 protein expression (FIG. 9B, at 12 and 24 hours), suggesting that H202 might be an important mediator for the cytokine-induced PRDX5 upregulation in human chondrocytes.

### Example 10

### Recombinant PRDX5 Protects Against Excitotoxic Brain Lesions

10.1 Animals and Drugs

Swiss mice of both sexes were used in this study. All experimental protocols and procedures were approved by our institutional review board and complied with guidelines of the Institut National de la Sante et de la Recherche Medicale (INSERM). Ibotenate (Tocris, Bristol, UK) was diluted in phosphate buffer saline (PBS) containing 0.01% acetic acid. S-bromowillardiine (Tocris), recombinant human PRDX5, mutated recombinant human PRDX5, catalase (Sigma, Saint Quentin Fallavier, France), catalase-polyethylene glycol (catalase-PEG ; Sigma), polyethylene glycol (PEG ; Sigma), and N-acetylcysteine (Sigma) were diluted in PBS.

Ibotenate activates N-methyl-D-aspartate (NMDA) and metabotropic glutamatergic receptors while S-bromowillardiine activates both alpha-3-amino-hydroxy-5-methyl-4-isoxazole propionic acid (AMPA) and kainate receptors (Largeron et al. 2001; Eur. J. Pharmacol. 424:189-94; Zorumski et al. 1991, Mol. Pharmacol. 40:45-51; Patneau et al., 1992, J. Neurosci. 12:595-606).

Recombinant PRDX5 contains the last 214 amino acids of the C-terminal part of the native human PRDX5 and contains the predicted peroxisomal addressing sequence as well as the catalytic site but is lacking the N-terminal predicted mitochondrial addressing sequence of the native molecule (Knoops et al., 1999, J. Biol. Chem. 274:30451-30548; Declercq et al., 2001, J. Mol. Biol. 311:751-9). PRDX5. Amino acid sequence of the mutated PRDX5 is similar to PRDX5 but with cysteine in position 47 replaced by a serine within the catalytic site of the molecule. Native and mutated PRDX5 were produced as previously described. N-acetylcysteine, catalase and catalase-PEG were used as reference anti-oxidant agents with hydrogen peroxide scavenging properties (Liu et al., 1989, Am. J. Physiol. 256:H589-93; He et al., 1993, Am. J. Physiol. 265:H252-6).

10.2 Excitotoxic Brain Lesions

Excitotoxic brain lesions were induced by injecting ibotenate (10 μg) or S-bromowillardiine (15 μg) into developing mouse brains, as previously described (see e.g. Tarahoui et al., 2001, Brain Pathol. 11:56-71; Largeron et al., 2001, Eur. J. Pharmacol. 424:189-94; Husson et al. 2002, Ann. Neurol. 51:82-92). Briefly, anesthetized mouse pups were kept under a warming lamp to maintain their body temperatures and were injected intra-cerebrally (into the neopallial parenchyma) with ibotenate or S-bromowillardiine on P5. Intracerebral injections were performed with a 25-gauge needle on a 50 μl Hamilton syringe mounted on a calibrated microdispenser. The needle was inserted 2 mm below the external surface of skin. The tip of the needle was in the frontoparietal area of the right hemisphere, 2 mm from the midline in the lateral-medial plane, and 3 mm from the bregma in the rostro-caudal plane. Histopathology confirmed that the tip of the needle always reached the periventricular white matter. Two 1 μl boluses of ibotenate or S-bromowillardiine were injected at a 20-s interval. The needle was left in place for an additional 20 s.

10.3 Experimental Groups

Pups from at least two different litters were used in each experimental group, and data were obtained from two or more successive experiments.

In a first set of experiments, P5 pups received an intracerebral injection of ibotenate immediately followed by an intraperitoneal injection with one of the following drugs diluted to a final volume of 5 μl: 0.1, 1, 10 or 20 mg/kg recombinant PRDX5; 10 mg/kg mutated recombinant PRDX5; 6,000, 60,000 or 600,000 units/kg catalase; 6,000, 60,000 or 600,000 units/kg catalase-PEG (1 mg=40,000 units); 15 mg/kg PEG; 0.25, 2.5, 25 or 250 mg/kg N-acetylcysteine; or PBS alone.

In a second set of experiments, P5 pups received an intracerebral injection of ibotenate followed by intraperitoneal administration of 10 mg/kg PRDX5 or PBS alone at three different times: immediately (T0h), 4 (T4h) or 8 (T8h) hours after ibotenate injection.

In the third set of experiments, P5 pups received an intracerebral injection of S-bromowillardiine immediately followed by an intraperitoneal injection with one of the following drugs diluted to a final volume of 5 μl: 10 mg/kg recombinant PRDX5; 600,000 units/kg catalase-PEG; 250 mg/kg N-acetylcysteine; or PBS alone.

10.4 Determination of Lesion Size

Mouse pups were sacrificed by decapitation 5 days (d) following the excitotoxic challenge. Brains were immediately fixed in 4% formalin and remained in this solution for 5 days. Following paraffin embedding, 16 μm thick coronal sections were cut. Every third section was stained with cresyl-violet. In theory, neocortical and white matter lesions can be defined by the maximal length of three orthogonal axes: the lateral-medial axis (in a coronal plane), the radial axis (also in a coronal plane, from the pial surface to the lateral ventricle) and the fronto-occipital axis (in a sagittal plane). In previous studies (Marret et al., 1995, J. Neuropathol. Exp. Neurol. 54:358-70; Gressens et al., 1997, J. Clin. Invest. 100:390-397; Husson et al. 2002, Ann. Neurol. 51:82-92), we showed an excellent correlation between the maximum size of the different diameters of the excitotoxic lesions. Based on these observations, we serially sectioned the entire brain in the coronal plane. This permitted an accurate and reproducible determination of the maximum sagittal fronto-occipital diameter (which is equal to the number of sections where the lesion was present multiplied by 16 μm) and was used as an index of the volume of the lesion.

10.5 Determination of In Vivo Cell Death

Eight and forty-eight hours after intracerebral injection of ibotenate plus intraperitoneal injection of 10 mg/kg recombinant PRDX5 or ibotenate plus PBS at P5, pups were sacrificed and brains were fixed in formalin and embedded in paraffin. Every third section was stained with cresyl violet, and sections adjacent to those containing the most affected areas were used for terminal transferase-mediated dUTP nick end labeling (Tunel), using an in situ cell death detection kit (Roche, Meylan, France). Deparaffinized sections were treated for 20 min at 37° C. with 20 mg/ml proteinase K and incubated for two min on ice with 0.1% Triton X-100. DNA strand breaks were identified by labeling of free 3′-OH termini with terminal deoxynucleotidyl transferase and fluorescein-labeled nucleotides for 60 min at 37° C. Incorporated nucleotides were detected using an anti-fluorescein antibody that was conjugated with alkaline phosphatase, using nitroblue tetrazolium and 5-bromo-4-chloro-3-indolyl phosphate toluidonium salt as the substrates. Tunel positive cells were counted in a 0.25 mm2 area in the neocortical layers and underlying white matter at the level of ibotenate-induced lesions. Ten nonadjacent fields (at least separated by 50 μm, to avoid counting twice the same nucleus) from five brains were studied in each group.

10.6 Primary Cultures of Neurons

pregnant E14.5 mice were killed under general anesthesia. Primary cultures of embryonic cortical neurons were prepared as previously described (de Lima et al., 1997, J. Comp. Neurol. 382:230-46). Briefly, the embryos were harvested, their brains were removed, and the cortex was dissociated both mechanically and chemically. Neurons were plated in 35-mm culture dishes previously coated with poly-DL-ornithine (30 μg/ml, Sigma) in Neurobasal® supplemented with 2 mM glutamine and B27 (Life Technologies, Cergy Pontoise, France), at a density of 8.105 cells/dish. Under these conditions, the neurons can survive and differentiate in the absence of an astroglial feeding layer. The cultures were maintained at 37° C. in a humidified 95% air-5% CO2 atmosphere. To inhibit proliferation of non-neuronal cells, 5 μM cytosine arabinoside (Sigma) was added after 3 to 6 days. The mean astroglial cell density in mature cultures was very low (less than 5%). We used only mature cultures (10-12 days in vitro) for excitotoxic challenge.

10.7 In Vitro Excitotoxic Challenge and Determination of Neuronal Cell Death

Neurons were incubated with 300 μM NMDA for 60 minutes in the presence of one of the following drugs or combination of drugs: 0.1, 1, 10 or 100 μM recombinant PRDX5; 10 μM dithiothreitol (DTT, Sigma); 10 μM recombinant PRDX5+10 μM DTT; or medium alone (control). After replacement of the medium, the neurons were left for an additional 8 hours in the presence of drugs, fixed in 4% paraformaldehyde, and finally stained with bis-benzimide (Hoechst 33452, 10 μg/ml; Sigma), which labels nuclear chromatin. An observer unaware of treatment conditions counted the nuclei that had features suggesting delayed cell death (namely, pycnosis, i.e., chromatin condensation or fragmentation), under a fluorescent microscope (UV-2A filter, Zeiss, Oberkochen, Germany, excitation 370 nm, emission>400 mm). Four to eight fields per plate, each containing 40-70 neurons, were examined. For each field, the ratio between the number of pycnotic nuclei and the total number of nuclei was calculated and used as an index of neuronal death. To take into account variations in cell viability across cultures, results were normalized for neuronal death with NMDA treatment under control conditions (i.e., without recombinant PRDX5 or DTT), taken as 100% excitotoxicity. Two to three plates were used for each experimental condition. Each experiment was performed at least twice.

10.8 Immunocytochemistry for PRDX5 on Cultured Neurons

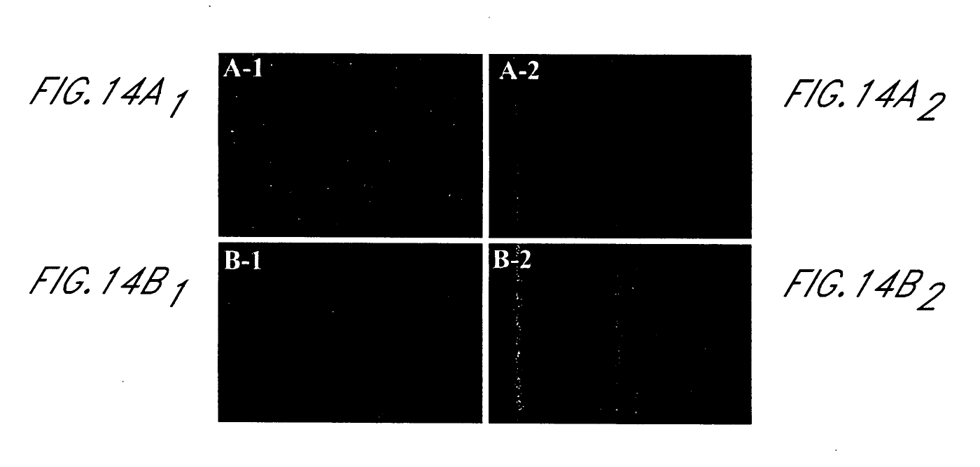

For immunostaining experiments, embryonic cortical neurons were cultured on coverslips. The cells were incubated with 100 μM recombinant PRDX5 or PBS for 0.5, 1, 2, 3, 6, or 8 h, rinsed with PBS and fixed with formalin-PBS (30 min). Fot immunostaining (FIG. 14), treated cells were rinsed three times with Tris-buffered solution (0.05 M, pH 7.6) containing 0.9% NaCl and 0.1% Triton X-100 (TBS-T), and immersed for 30 min. in TBS-T containing 10% nonfat milk. Coverslips were then incubated overnight with rabbit antihuman PRDX5 diluted 1:200 in TBS-T containing 1% nonfat milk (Martin et al., 2000, Neurobiol. Dis. 7:169-91), followed by an incubation with fluorescein isothiocyanate conjugated donkey antirabbit IgG (Jackson Immunosearch Laboratories Inc. West Grove, Pa., USA) diluted 1/20 in TBS-T for 1 h. After each incubation, cells were washed twice for 10 min with TBS-T. The coverslips were mounted in Mowiol containing antifading reagent [1,4-diazabicyclo(2.2.2)octane, 25 mg/ml; Sigma] and 4′,6-diamidino-2′phenylindole (DAPI) (50 μg/ml; Roche Basel, Switzerland) for nuclear staining. The coverslips were examined for fluorescein and DAPI by fluoresence microscopy with standard filters.

10.9 Statistical Analysis

All variables were found normally distributed based on Skewness and Kurtois analyses (Tsuji et al., 2000, Pediatr. Res. 47:79-83). Most data were analyzed with a Student's t-test or a univariate analysis of variance (ANOVA)(GraphPad Prism version 3.03 for Windows, Graphpad Software). In ANOVA analyses, when group interaction was found to be significant, a Dunnett's multiple comparison test was performed. In the subset of experiments where lesion size was evaluated at different timepoints after ibotenate injection, results were studied using ANOVA with Treatments and Age (time elapsed after injection) as between-subject factors. When Age by Treatment interaction was significant, we conducted comparisons between treatment groups and control (PBS) group using Dunnett's post-hoc test.

Results

10.10 Clinical Manifestations and Mortality

Overall, mortality was low (<3%) in pups receiving intracerebral ibotenate or S-bromowillardiine injections. All pups injected with ibotenate or S-bromowillardiine displayed tonic-clonic convulsions within the first 24 h following the excitotoxic challenge. Co-treatment with intraperitoneal PRDX5, catalase, catalase-PEG or N-acetylcysteine did not modify mortality significantly (<3% in all experimental groups) nor did it alter the incidence, severity, or phenotype of convulsions.

10.11 Excitotoxic Lesions and Neuroprotection of Recombinant PRDX5

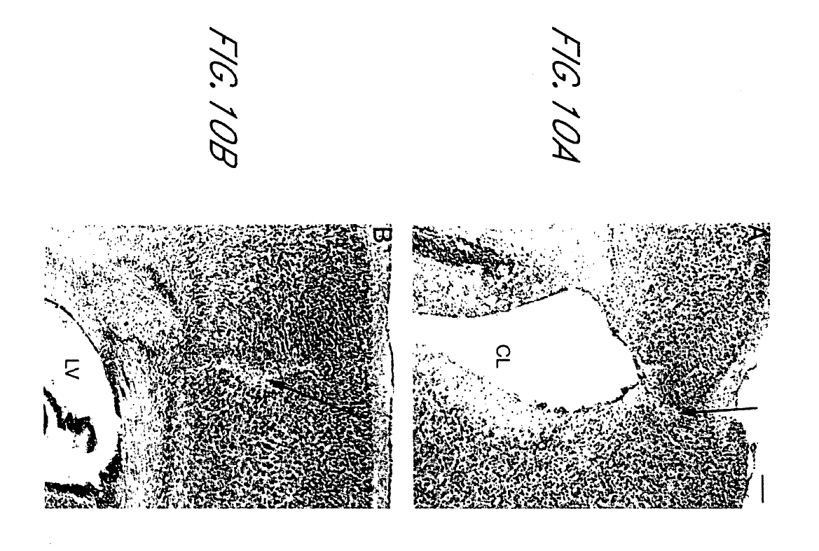

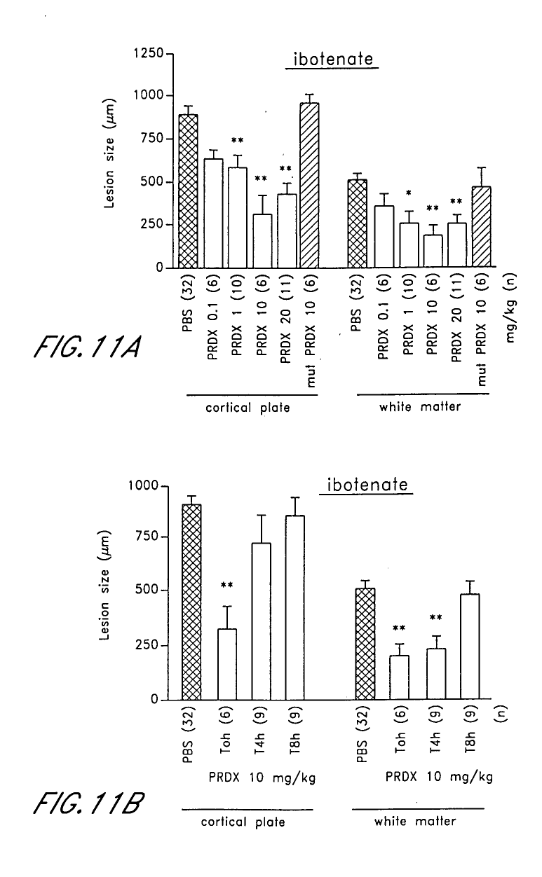

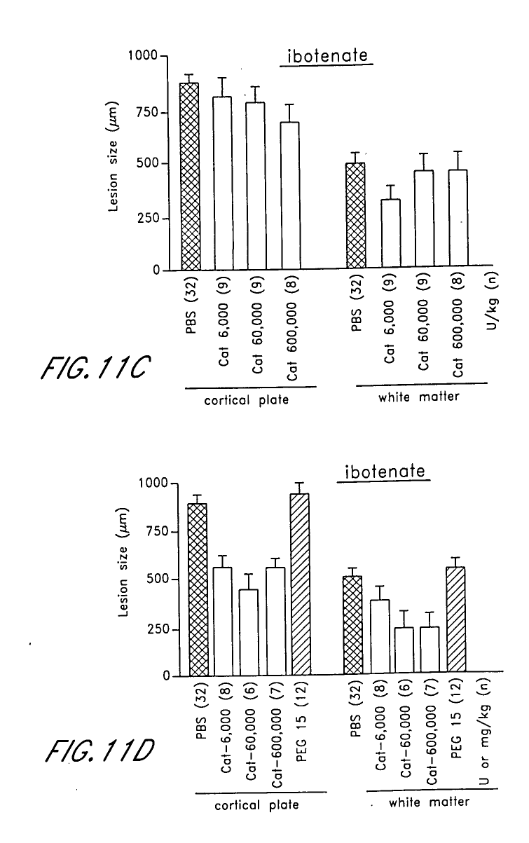

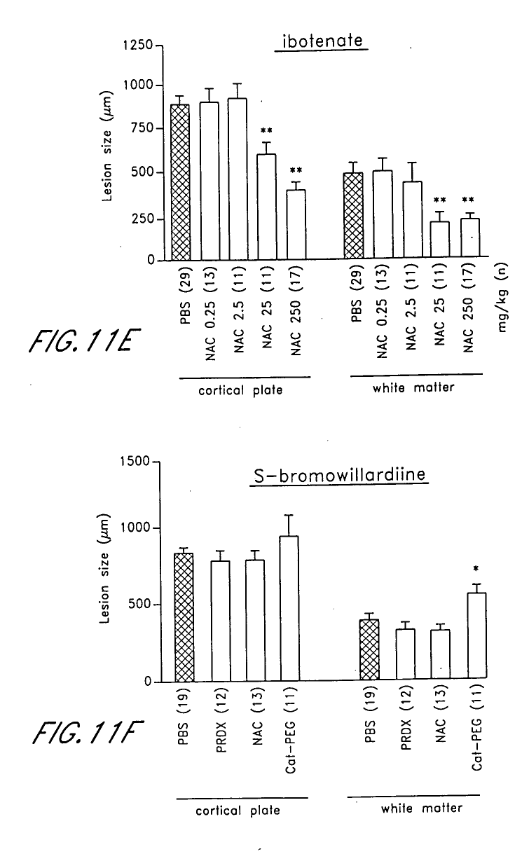

Pups injected intracerebrally with ibotenate and intraperitoneally with PBS on P5 developed cortical lesions and periventricular white matter cysts (FIGS. 10A and 11A). The cortical lesion was typical, with severe neuronal loss in all neocortical layers and almost complete disappearance of neuronal cell bodies along the axis of ibotenate injection.

Intraperitoneal administration of recombinant PRDX5 immediately after ibotenate produced a dose-dependant protection against the ibotenate-induced cortical plate and white matter lesions (FIGS. 10B and 11A). In contrast, mutated recombinant PRDX5 had no detectable protective effect against ibotenate-induced lesions (FIG. 11A). When intraperitoneal injection of recombinant PRDX5 followed the excitotoxic challenge, neuroprotection was a function of time. Protection of the cortical plate was observed in groups receiving recombinant PRDX5 immediately after ibotenate while white matter protection was observed in groups receiving recombinant PRDX5 within the first 4 hours after ibotenate administration (FIG. 11B).

Co-treatment with intracerebral ibotenate and intraperitoneal catalase-PEG or N-acetylcysteine, but not catalase, mimicked the neuroprotective effects of recombinant PRDX5 against excitotoxic brain lesions (FIGS. 11C-E).

Intracerebral injection of S-bromowillardiine also produced cortical plate lesions and periventricular white matter cysts (FIG. 11F). Intraperitoneal administration of recombinant PRDX5, catalase-PEG or N-acetylcysteine had no detectable protective effect on the S-bromowillardiine-induced brain lesions (FIG. 11F).

10.12 Effects of Recombinant PRDX5 on Excitotoxic Neuronal Cell Death

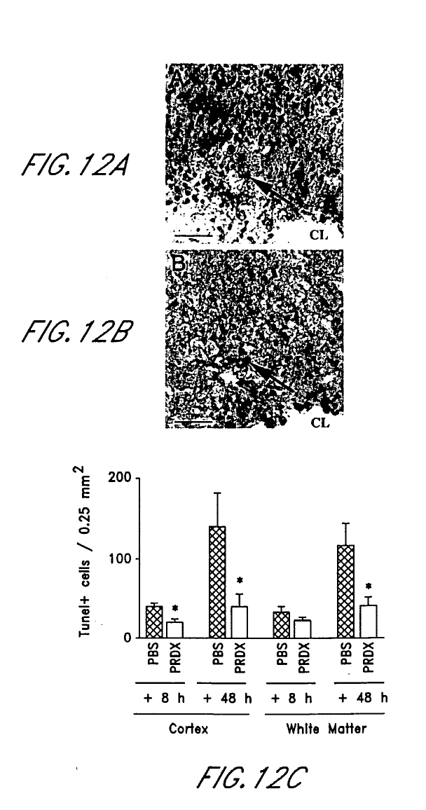

Tunel staining performed at 8 and 48 hours following ibotenate injection showed that recombinant PRDX5 induced a significant reduction of cortical plate (at both studied time-points) and white matter (at 48 hours post-ibotenate) cell death (FIG. 12).

10.13 Effects of Recombinant PRDX5 on In Vitro Neuronal Cell Death

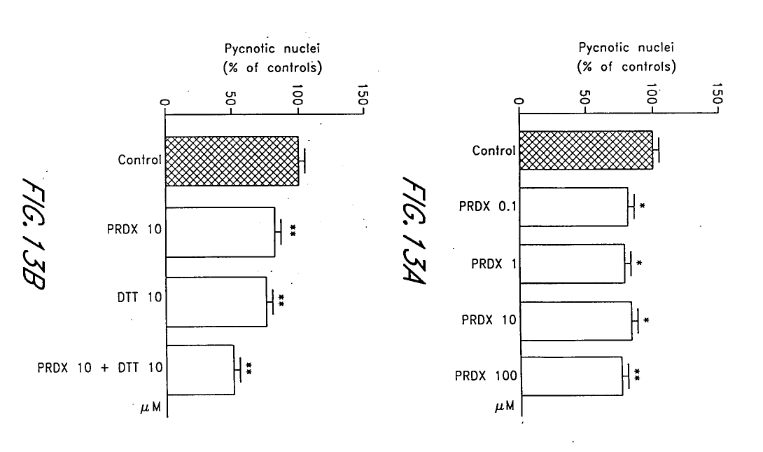

When compared to cultures treated with PBS (controls), exposure to recombinant PRDX5 induced a moderate but significant reduction of NMDA-induced neuronal cell death (FIG. 13A). Interestingly, the co-treatment with equimolar recombinant PRDX-5 and DTT (an electron donor) induced a larger reduction of NMDA-induced neuronal cell death when compared to PRDX-5 alone or DTT alone (FIG. 13B).

10.14 Intracellular Localization of Exogenous Recombinant PRDX5 in Neuronal Cultures

A rabbit polyclonal antibody directed to PRDX5 was used to localize recombinant PRDX5 in neuronal cultures. Immunoreactivity was intense in cells incubated with recombinant PRDX5 for 30 min to 8 h (FIG. 14A-1), and weak in cells treated with PBS only (FIG. 14 B-1). Immunoreactivity in PBS-treated cells reflected endogeneous PRDX5. In cultures treated with PRDX5, cells with neuronal phenotype showed the most intense labeling.

TABLE 1
Homologies of the B18 proteins (162 amino
acid) with other proteins

| | | Identity (%) |
|---|---|---|
| Name | NCBI ID | Homology (%) |
| Membrane protein | 1652859 | 57/129(44%) |
| (synechocystis sp.) | | 81/129(62%) |
| Peroxisomal-like protein | 2769700 | 56/176(31%) |
| (Aspergillus fumigatus) | | 90/176(50%) |
| Haein HI0572 hypothetical | 1723174 | 53/146(36%) |
| protein(Haemophilus | | 80/146(54%) |
| influenzae) | | |
| PMP20 (Schizosaccharomyces | AJ002536 | 54/161(33%) |
| pombe) | | 85/161(52%) |
| Peroxisomal membrane | 130360 | 59/170(34%) |
| protein A (PMP 20) (Candida | | 89/170(51%) |
| boidinii) | | |
| Peroxisomal membrane | 130361 | 58/170(34%) |
| protein B (PMP 20) (Candida | | 88/170(51%) |
| boidinii) | | |
| Putative peroxisomal | 1709682 | 41/138(29%) |
| protein PMP from yeast | | 72/138(51%) |
| (Saccharomyces cerevisiae) | | |
| Alkylhydroperoxide | P26427 | 36/126(28%) |
| reductase C22 protein | | 58/126(45%) |
| (Escherichia coli) | | |

TABLE 2

| Name | Access NO | Identity |
|---|---|---|
| Human mRNA down-regulated in | U82616 | 259/263 (98%) |
| cells infected by adenovirus 5 | | |
| Human mRNA down-regulated in | U82615 | 300/321 (93%) |
| cells infected by adenovirus 5 | | |

1. An amino acid sequence having more than 70% homology with the sequence SEQ ID NO: 2.

2. The amino acid sequence according to claim 1, having more than 85% homology with the sequence SEQ ID NO: 2.

3. The amino acid sequence according to claim 1, having more than 95% homology with the sequence SEQ ID NO: 2.

4. The amino acid sequence according to claim 2, having more than 95% homology with the sequence SEQ ID NO: 2.

5. An amino acid sequence corresponding to SEQ ID NO: 2 or a portion thereof selected from the group consisting of the sequences comprised between:
the glutamic acid in position 13 and the glutamic acid in position 27,
the alanine in position 26 and the leucine in position 36,
the alanine in position 42 and the glutamic acid in position 57,
the glutamic acid in position 57 and the valine in position 69,
the valine in position 80 and the leucine in position 97,
the arginine in position 95 and the leucine in position 112,
the serine in position 118 and the serine in position 129,
the valine in position 137 and the threonine in position 150,
the glutamic acid in position 13 and the cysteine in position 47,
the glutamic acid in position 13 and the glycine in position 38,
the leucine in position 36 and the cysteine in position 47, and
the threonine in position 150 and the leucine in position 162.

6. A pharmaceutical formulation in an orally administrable dosage form, comprising:
(a) the amino acid sequence according to claim 1, or a pharmaceutically acceptable salt or derivative thereof, and
(b) possibly a pharmaceutically acceptable reductant and/or electron donor.

7. A method of treating neurotoxic injury in a patient suffering from said injury by administering to said patient a composition comprising the amino acid sequence according to claim 1, its pharmaceutically acceptable salts or derivatives and pharmaceutically acceptable esters, and a pharmaceutically acceptable carrier, wherein said compound is present in said composition in an amount effective to treat said neurotoxic injury.

8. A method of decreasing the effect of excitotoxic injury in a patient, having said injury, comprising administrating to said patient a composition comprising the amino acid sequence according to claim 1, its pharmaceutically acceptable salts or derivatives and pharmaceutically acceptable esters, and a pharmaceutically acceptable carrier, wherein said compound is present in said composition in an amount effective to treat said excitotoxic injury in said patient.

9. The method according to claim 8, wherein said excitotoxic injury is caused by oxidative stress.

10. The method according to claim 9, wherein said excitotoxic injury is osteoarthritis.

11. The method according to claim 9, wherein said excitotoxic injury affects neuronal cells.

12. The amino acid sequence according to claim 1, produced in yeast.
